(19) United States
(12) Patent Application Publication
(10) Pub. No.: US 2026/0256604 A1
(43) Pub. Date: Sep. 3, 2026

(54) BIORESORBABLE STENTS AND ASSOCIATED SYSTEMS AND METHODS

(71) Applicant: Nativene, Inc., Accord, MA (US)

(72) Inventors: Abha Chinubhai, Fremont, CA (US); Jeffrey Michael Elkins, Woodside, CA (US); Thomas Edwin Hancock, Hingham, MA (US); Neema Hekmat, Benicia, CA (US)

(21) Appl. No.: 19/553,294

(22) Filed: Feb. 28, 2026

Related U.S. Application Data
(60) Provisional application No. 63/764,581, filed on Feb. 28, 2025.
(60) Provisional application No. 63/896,359, filed on Oct. 9, 2025.

(51) Int. Cl. A61F 2/86

(52) U.S. Cl. CPC A61F 2/86; A61F 2210/0004

(57) ABSTRACT

Disclosed is a bioresorbable stent devices and associated systems and methods. In some embodiments, a stent device can include a tubular frame with a first open end, a second open end, a lumen extending therebetween, and a plurality of struts defining the lumen. The struts can be made from a magnesium alloy and a predefined maximum grain size. The tubular frame can be configured to elastically deform and withstand at least 40 percent compression associated with dynamic vein movement. The stent device can further include one or more anchor protrusions that are configured to engage tissue of an inner vein wall to resist stent migration. The stent device can have a degradation time ranging from three to six months after implantation within a vessel.

### CROSS-REFERENCE TO RELATED APPLICATION(S)

This application claims priority to and benefit from to U.S. Provisional Patent Application No. 63/896,359, filed Oct. 9, 2025, and U.S. Provisional Patent Application No. 63/764,581, filed Feb. 28, 2025, the contents of which are incorporated herein by reference in their entireties.

### TECHNICAL FIELD

The present technology relates generally to medical devices and, more particularly, to bioresorbable stents and associated systems and methods.

### BACKGROUND

Stents are medical implant devices used for supporting, reopening, and/or expanding narrowed, blocked, and/or otherwise compromised blood vessels or other tubular structures in the body to restore or maintain adequate flow. Stents can be used as effective interventional devices for treating various conditions requiring vascular support and patency maintenance for arteries or veins. The conditions can be due to intrinsic or extrinsic obstructions or occlusions. Venous conditions that can be treated with stents implantations include, for example, venous thromboembolism (VTE) (e.g., deep vein thrombosis (DVT)), post-thrombotic syndrome (PTS) and non-thrombotic iliac vein lesions (NIVL). Because veins and arteries differ in anatomy, pressure dynamics, flow patterns, and disease processes, their stents have different mechanical demands and clinical risks. For example, due to the anatomy of veins, vein stents require greater radial strength, flexibility, compression resistance, and antimigration and antithrombotic features compared to arterial stents.

Standard metal stents are typically permanent implants that can be effective in maintaining vessel patency. However, these stents can present long-term complications including chronic inflammation, early or late stent thrombosis, in-stent restenosis, and complications associated with future interventions at the stent site. Studies have shown that in as many as half of patients, patency of a conventional stent is lost at just one year following the implantation due to in-stent re-thrombosis resulting in restenosis (i.e., narrowing of the stent). Further, conventional re-stenting of permanent stents can further narrow the lumen (as the previous stent typically cannot expand further) causing increase in flow, shear rates, and in turn further increasing risk of earlier occlusion, while stent on stent contact is a known risk factor for stent corrosion and fracturing. Further, stent migration can raise thrombotic risk and obstruct future vascular access (e.g., if a stent is covering an ostia of a branching lumen), with no reliable way to reposition or remove the device short of open surgery.

### BRIEF DESCRIPTION OF THE DRAWINGS

Many aspects of the present disclosure can be better understood with reference to the following drawings. The components in the drawings are not necessarily to scale. Instead, emphasis is placed on clearly illustrating the principles of the present disclosure.

FIG. 1A is an isometric view of a stent device configured in accordance with some embodiments of the present technology.

FIGS. 1B and 1C are side views of a portion of the stent device of FIG. 1A in a low-profile state and an expanded state, respectively, in accordance with some embodiments of the present technology.

FIG. 1D is a cross-sectional view of the stent device of FIG. 1A.

FIG. 2 is an isometric view of the stent device of FIGS. 1A-1D positioned within a vessel in accordance with some embodiments of the present technology.

FIGS. 3A and 3B illustrate the stent device of FIGS. 1A-1D at different stages of deployment in accordance with some embodiments of the present technology.

FIG. 4A is a side view of a stent device with varying strut thickness in accordance with some embodiments of the present technology.

FIG. 4B is a side view of a stent device with a tapered diameter in accordance with some embodiments of the present technology.

FIG. 4C is a side view of a stent device with a supporting spine and varying structural patterns in accordance with some embodiments of the present technology.

FIGS. 5A-5C are isometric views of stent devices having varying strut structures in accordance with some embodiments of the present technology.

FIG. 6A is an isometric view of the stent device of FIGS. 1A-1D with marker elements in accordance with some embodiments of the present technology.

FIG. 6B is an enlarged isometric view of a portion of the stent device of FIG. 6A.

FIGS. 7A and 7B are isometric views of embodiments strut configurations for stent devices in accordance with some embodiments of the present technology.

FIG. 8 is a side view of the stent device of FIGS. 1A-1D with a filter in accordance with some embodiments of the present technology.

FIG. 9 is a flow diagram of a method for delivering a bioresorbable stent in accordance with some embodiments of the present technology.

### DETAILED DESCRIPTION

### Overview

Described herein are bioresorbable, magnesium (Mg) alloy-based, stent devices (also referred to as stents) for treatment of cardiovascular conditions affecting veins, as well as associated systems and methods. The described stents are made from a Mg-alloy-based material that can gradually degrade and be absorbed by the body after maintaining vessel patency during treatment (e.g., within 3-6 months). The bioresorbable stents can eliminate long-term foreign material, restoring natural vessel motion, restoring smooth inner lumen and reducing late stent-related complications (such as in-stent thrombosis, fracture or chronic inflammation). Additionally this allows for the vein to return to its natural function as a capacitance vessel, which is critical to allowing effective pumping of blood by the venous system and draining of the venous blood reservoir. Furthermore, the return of a smooth inner lumen (by way of a resorbed stent) reduces in-stent thrombosis by smoothing flow lines and reducing platelet activation. The described stent devices can also be applied for relining previously implanted permanent metal stents, to prevent permanent further constriction of the flow lumen (since the resorbable inner stent will resorb). The described stent devices can also be applied for relining previously implanted resorbable stents as there would be no compromise to the flow lumen and will provide a smoother lumen when both stents have fully resorbed, both factors acting to further mitigate re-thrombosis risk.

More specifically, the disclosed vein stents are configured for implantation in the inflow vessels (veins) of the venous systems, such as the femoral, tibial, popliteal, fibular, saphenous, or femoral veins (e.g., profunda femoris) as well as iliofemoral vessels. Due to the specific anatomy of these veins, treatment of conditions using vein stent mechanisms presents unique challenges. For example, because the inflow veins can traverse through segments of flexion points in the body (e.g., inguinal ligament, adductor canal, knee), vein stents must balance high, durable radial force with atraumatic conformability to maintain patency under variable extrinsic loads and joint motion. The stents must also accommodate compression, elongation, and torsion with high fatigue resistance, promoting crush recoverability and kink resistance while minimizing foreshortening and fracture risk. The disclosed Mg-alloy vein stents address these challenges with a combination of a scaffolding structure and the Mg-alloy material that enable the stent to operate as a spring configured to collapse and rebound when positioned in situ. For example, the disclosed stent can be compressed or otherwise moved to a low-profile state that is half its original diameter or cross-sectional area by external forces and still maintain structural integrity and adequate lumen to support blood flow, then recover to its intended shape once the load is removed. The described vein stent also provides for a high (e.g., up to 5-6-fold) expansion to allow a vein stent to achieve firm circumferential apposition in compliant, variably shaped veins to reduce recoil and migration while maximizing lumen restoration and patency under external compression.

The described stents are therefore configured to deliver high, durable radial resistive force (e.g., crush strength) or chronic outward force to resist external compression from fascia, bone, inguinal ligament, and muscles while maintaining patency in compliant, noncircular veins. The described stents also provide crush recoverability, kink resistance, and flexibility to tolerate compression, elongation, and torsion across flexion points without fracturing or foreshortening. For example, rush and fracturing by inguinal ligament and pubis bone cause a challenge for vein stents. Further, to prevent mobility and migration in low-shear venous flow, the described vein stents include tailored anchoring mechanisms that preserve physiological curvature and avoid extension across major junctions or valves.

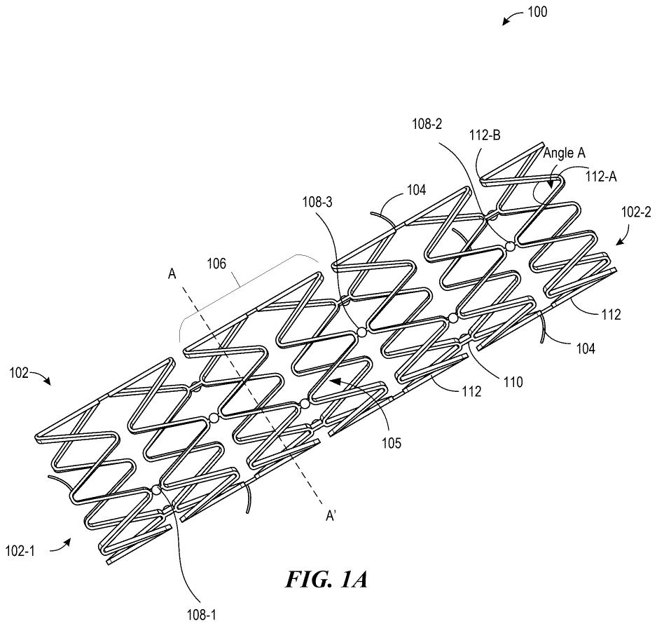

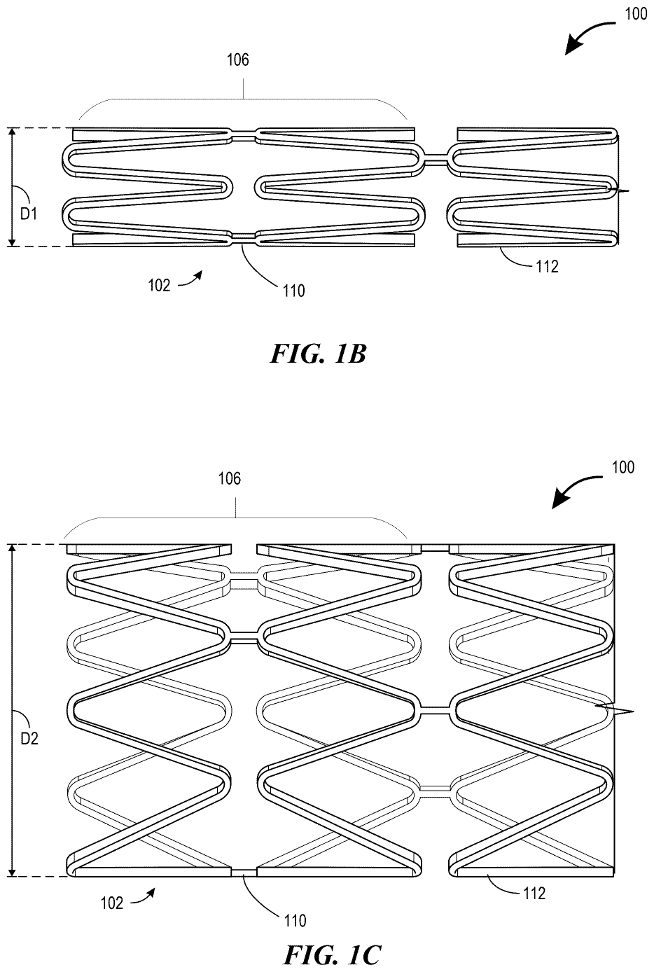

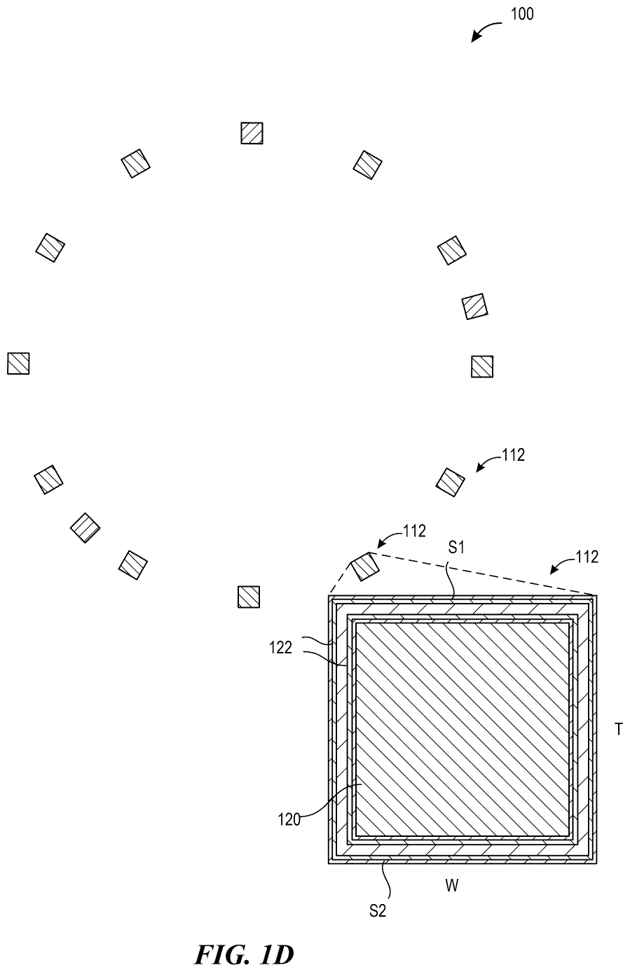

Specific details of several embodiments of the present technology are described herein with reference to FIGS. 1-9. The present technology, however, can be practiced without some of these specific details. In some instances, well-known structures and techniques often associated with vein stent devices have not been shown in detail so as not to obscure the present technology. The terminology used in the description presented below is intended to be interpreted in its broadest reasonable manner, even though it is being used in conjunction with a detailed description of certain specific embodiments of the disclosure. Certain terms can even be emphasized below; however, any terminology intended to be interpreted in any restricted manner will be overtly and specifically defined as such in this Detailed Description section.

The accompanying Figures depict embodiments of the present technology and are not intended to be limiting of its scope. The sizes of various depicted elements are not necessarily drawn to scale, and these various elements can be arbitrarily enlarged to improve legibility. Component details can be abstracted in the Figures to exclude details such as the position of components and certain precise connections between such components when such details are unnecessary for a complete understanding of how to make and use the present technology. Many of the details, dimensions, angles, and other features shown in the Figures are merely illustrative of particular embodiments of the disclosure. Accordingly, other embodiments can have other details, dimensions, angles, and features without departing from the spirit or scope of the present technology.

FIGS. 1A-1D illustrate various views of a stent device 100 configured in accordance with some embodiments of the present technology. Specifically, FIG. 1A is an isometric view of a stent device 100, FIG. 1B is a side view of a portion of the stent device 100 in an unexpanded state (also referred to as a “first state,” a “collapsed state,” a “contracted state,” a “low-profile state,” and the like), FIG. 1C is a side view of the portion of the stent device 100 in an expanded state (also referred to as a “second state,” a “deployed state,” and the like), and FIG. 1D is a cross-sectional view of the stent device 100 taken along line A-A′ in FIG. 1A. As shown in FIG. 1A, the stent device 100 includes a tubular frame 102 (also referred to as a “scaffold”) formed by a plurality of interconnected struts 112 (e.g., wires, a laser cut tube). The tubular frame 102 has a first open end 102-1, a second open end 102-2, and a lumen 105 extending between the first open end 102-1 and the second open end 102-2. The tubular frame 102 is configured to elastically deform, for example, after being implanted in a vein. The struts 112 include a bioresorbable Mg alloy having a concentration of Mg of at least 95 percentage weight and a maximum grain size of 10 micrometers. Due to, for example, the strut patterns and properties of the strut (e.g., thickness and the alloy material/coatings), the tubular frame 102 is configured to withstand at least 40 percent compression (e.g., at least 40 percent, at least 50 percent, or at least 60 percent compression) associated with dynamic movement of the vein. Such high compression is needed in vein stents to resist external compression from surrounding tissues and maintain lumen patency in low-pressure, collapsible venous vessels. The stent device 100 also includes one or more anchor protrusions 104 coupled to at least one of the struts 112 and extending outwardly away from the lumen 105. The anchor protrusions 104 are configured to contact (e.g., engage or penetrate) an inner wall of the vein to resist migration of the stent device during and after implant. The resistance to migration is especially important in veins because they are low-pressure, highly compliant vessels with less intrinsic wall recoil causing low frictional resistance.

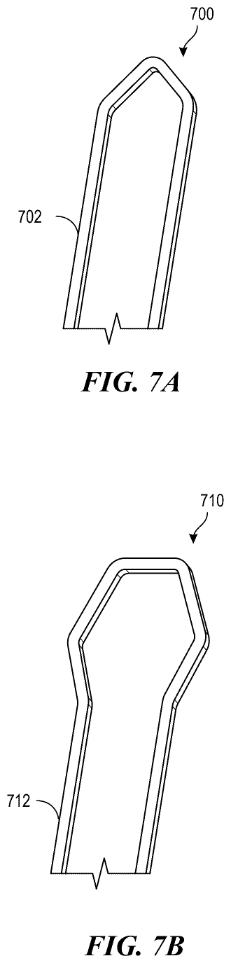

The struts 112 are arranged in strut patterns 106 having peak regions (e.g., peak regions 112-A and 112-B) extending sequentially in opposing directions along the length of the tubular frame 102 (e.g., in a zigzag manner along the circumference of the tubular frame 102). The strut patterns 106 are coupled with each other at the peak regions 112-A and 112-B by connector regions 110. In the embodiment of FIG. 1A, the peak regions 112-A and 112-B have a rounded shape (e.g., a rounded V-shape) configured to reduce stress at the peak regions. For example, the peak regions 112-A and 112-B can define an angle of at least 30 degrees, or at least 40 degrees or of at least 50 degrees between adjacent struts 112. The shape of the strut patterns 106 can be tailored to provide desired structural properties, as is discussed with respect to FIGS. 7A and 7B.

The stent device 100 including the struts 112, and in some embodiments, the anchors protrusions 104 are made of a Mg alloy having a concentration of Mg of at least 90 percentage weight (weight %) (e.g., at least 90 weight %, at least 95 weight %, at least 98%, or at least 99 weight %). A higher Mg content in the Mg alloy can accelerate corrosion and degradation when the stent is implanted in situ and thereby shorten the radial support duration of the bioresorbable stent. The Mg in the Mg alloy has a maximum grain size of 10 micrometers, 5 micrometers, 1 micrometer or 0.1 micrometers. Mg micro grain structures can improve initial radial/compressive strength, crimp ability, and deformation uniformity of the stent device 100. Micro grain structures also support uniform corrosion and slower loss of support while degrading. Further, the micro grain structures of the Mg alloy can have a specific grain orientation.

In some embodiments, a Mg grain structure oriented in a particular direction can improve radial strength, crimpability, and fatigue resistance, and can produce more uniform corrosion, stabilizing radial support over time. For example, orienting the Mg micro grain structures so the basal planes (e.g., the flat plane at a base of a Mg crystal structure) do not resist the expansion of the tubular stent around its circumference can cause the stent to have more resistance to circumferential deformation. The orientation of the Mg micro grain structures can cause higher initial radial strength and higher spring-back capability (e.g., causing a higher compression rate). The grain structure orientation can be achieved during manufacturing by cold drawing which introduces pronounced texture (preferred grain orientation), typically aligning basal planes due to slip and twinning during deformation. Subsequent heat treatment (recovery/recrystallization/annealing) can modify or partially randomize that texture by new grain formation and growth, with the extent depending on temperature, time, and prior strain. For example, the Mg alloy can be treated during manufacture with cold drawing and heat treatment to orient the grain structures in a particular manner to increase the elasticity of the tubular frame. In certain stent designs, however an isotropic material performance may be desired, and thus, achieved by not biasing the orientation of the grains, and instead achieving randomness in the grain structure. This may benefit certain properties such as increasing resistance to cracking or crack propagation.

The structural and composition properties of the disclosed stent device are configured to provide performance characteristics that ensure sustained vessel patency under external forces and stent device's ability to regain luminal diameter after compression. These properties include, for example, crush resistance (e.g., a force required to radially compress the stent to a specified diameter reduction), diameter recovery (e.g., a percent return toward original diameter after removal of a radial compression), and foreshortening (e.g., a percent change in stent length from a collapsed state to an expanded state). Table 1 below summarizes performance characteristics of stent devices with varying structural properties, as shown by experimental results. For example, increasing the length of links between the strut patterns decreases foreshortening. Also, the crush resistance (relational to stiffness) and diameter recovery post compression can be modulated using varying strut patterns and/or thicknesses.

TABLE 1
Performance Characteristics of Mg Alloy Stent
Devices with Varying Structural Properties.

| | Stent 1 | Stent 2 | Stent 3 | Stent 4 |
|---|---|---|---|---|
| Strut Thickness | 457 | 457 | 457 | 150 |
| (micrometers) | | | | |
| Structural | Six peaks; | Six peaks; | 24 peaks; | 24 peaks; |
| Features | long peak to | long strut to | short peak to | short peak to |
| | peak links | strut links | peak links | peak links |
| Stent | 6.7 | 6.7 | 14.7 | 13 |
| Foreshortening | | | | |
| (%) | | | | |
| Crush | 0.17 | 0.15 | 0.14 | 0.25 |
| Resistance | | | | |
| (N/mm) | | | | |
| Diameter | 81.3 | 82.4 | 82.4 | 99.5 |
| Recovery (%) | | | | |

In some embodiments, the Mg alloy further includes one or more additional metals. For example, the Mg has alloy up to 10 weight % of manganese, zinc, and/or calcium (e.g., up to 10 weight %, up to 5 weight %, up to 2 weight % or up to 1 weight % of zinc and/or calcium). In some embodiments, the Mg alloy does not include rare earth metals. Rare earth metals include lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), and lutetium (Lu). In addition, the rare earth family customarily includes scandium (Sc) and yttrium (Y). In some embodiments, the Mg alloy includes one or more rare earth metals (e.g., WE43, AZ31, ZK60). In some embodiments, the Mg alloy includes up to 5 weight % of other elements to provide certain properties. For example, lithium (Li), cadmium (Cd) or thallium (TI) can support expansion of the tubular frame without strut fracturing (e.g., increase flexibility of the Mg alloy); aluminum (Al), zinc (Zn), calcium (Ca), silver (Ag), Cesium (Ce), gallium (Ga), nickel (Ni), copper (Cu), and thorium (Th) can improve the strength and plasticity of the Mg alloy; tin (Sn), antimony (Sb), bismuth (Bi), and lead (Pb) can ameliorate the strength of the Mg alloy; and aluminum (Al), indium (In), manganese (Mn), zinc (Zn), zirconium (Zr) and yttrium (Y) can enhance corrosion resistance of the Mg alloy.

In alternative embodiments, the stent device 100 is made of iron alloys (e.g., iron alloy including lead, carbon, and/or manganese) or zinc alloys (e.g., zinc alloy including manganese, calcium, or magnesium). In some embodiments, the stent device 100 can be made of one or more resorbable materials selected from calcium compounds, bioactive glass materials, polylactic acids including poly-l-lactic acid (PLLA), Poly(D-lactic acid) or poly(D-lactide) (PDLA), Poly(D,L-lactic acid) or poly(D,L-lactide) (PDLLA), Polyglycolic acid (polyglycolide) (PGA), Poly(lactic-co-glycolic acid) (PLGA), Polycaprolactone (PCL), Poly(3-hydroxybutyrate) (PHB), Poly(3-hydroxybutyrate-co-3-hydroxyvalerate) (PHBV), poly(sebacic anhydride), poly(adipic anhydride), poly(ortho ester), tyrosine-defined polycarbonates, trimethylene carbonate copolymers or Polyethylene glycol (PEG).

The stent device has a degradation time ranging from three to six months, three to four months, or up to three months after implanted within the vein. In some embodiments, the degradation time can be shorter (e.g., 2-4 weeks) or significantly longer (e.g., more than a year). The degradation time can be tailored to maintain structural integrity during endothelialization and the critical healing period followed by progressive dissolution over a predetermined timeline. Several factors of the stent device 100 govern the degradation time. The base material of the struts 112 is a primary (e.g., the Mg alloy) factor as well as any coatings on the strut surfaces (e.g., as described with respect to FIG. 1D). Mg degrades through a corrosion process influenced by physiological fluid exposure, with degradation products including magnesium hydroxide and hydrogen gas. Polymer-based components (coatings) degrade through hydrolysis, with breakdown into smaller molecular chains and eventually into biocompatible byproducts. In some embodiments, in vivo testing has shown that the disclosed stent device with one or more coatings that slow down the biodegradation of the Mg alloy can be incorporated (ingrown) faster into the vein as compared to conventional stent devices made of bare metal (e.g., bare nickel-titanium alloys). The faster incorporation into the vein can be attributed to the bioinert properties of the coating, and being more bioinert than bare metal. The fast incorporation ensures that stent migration risk is minimized and also protects against embolic risk, which would otherwise be a concern if the coating were to fully degrade before being incorporated into the vein (leaving the Magnesium backbone exposed to blood for potential fragmentation). Also, strut geometry affects the degradation time. For example, thicker, wider, or less porous struts slow water ingress and hydrolysis/corrosion thereby extending degradation, while thinner designs accelerate it. Structural factors—including cell pattern, connectors, and surface area-to-volume ratio—can affect local stress, crack initiation, and fluid exposure, and coating integrity and porosity, which can hasten or slow breakdown. For example, test results have shown that coatings having larger pore diameters (e.g., above 1 micrometer) and denser pores have been shown to provide higher reactivity to in vitro media (e.g., Hank's Balanced Salth solution) as compared to coatings with no discernable pores (e.g., no pores having diameter above 1 micrometer. The pore sizes and density of coatings were analyzed with scanning electron microscoping (SEM) imaging.

The anchor protrusions 104 in the embodiment of FIG. 1A (e.g., anchor barbs or pins) extend outwardly away from the outer surface of the tubular frame to provide axial stability or resistance against migration. The anchor protrusions 104 are configured to contact an inner wall of the vein to resist migration of the stent device 100 during and after implantation. The anchor protrusions 104 can be oriented radially outward in the same direction or different directions, with the same angle or different angles. The anchor protrusions 104 can be placed in varying patterns along the stent, for example, in circumferential, axial, helical and complex (e.g., zig zag) patterns. These patterns may be repeated along the stent (e.g., multiple circumferential rings of anchors) or in mixed patterns (e.g., circumferential at proximal end of stent and helical pattern for the rest of the stent length). The anchor protrusions 104 can be straight or curved barbs or pins. The tips of the anchor protrusions 104 may be unsharpened or sharpened. If sharpened, they could be shaped in any of the following configurations or combinations thereof: beveled tips, double-beveled tips, triple-beveled tips, conical tips, tapered tips, lanced tips, trocar tips, lancet tips, taper-flat hybrid tips, serrated tips, micro-serrated tips, radially symmetric tips, multi-facet tips, hollow-ground tips, concave tips, laser-cut tips, etched micro-tips, blunt tips, atraumatic tips, composite tips, modular tips, directional tips, side-cutting tips, helical tips, screw-type tips, retractable tips, and/or shielded tips. The stent device 100 can include, additionally or alternatively to the anchor protrusions, other types of securing mechanisms such as strut surface texture/roughness, bumps, edges, ribs, loops, rings, etc. Such securing materials can restrain the stent device migration by providing additional friction and/or engaging (e.g., pinching) the inner wall of a vein.

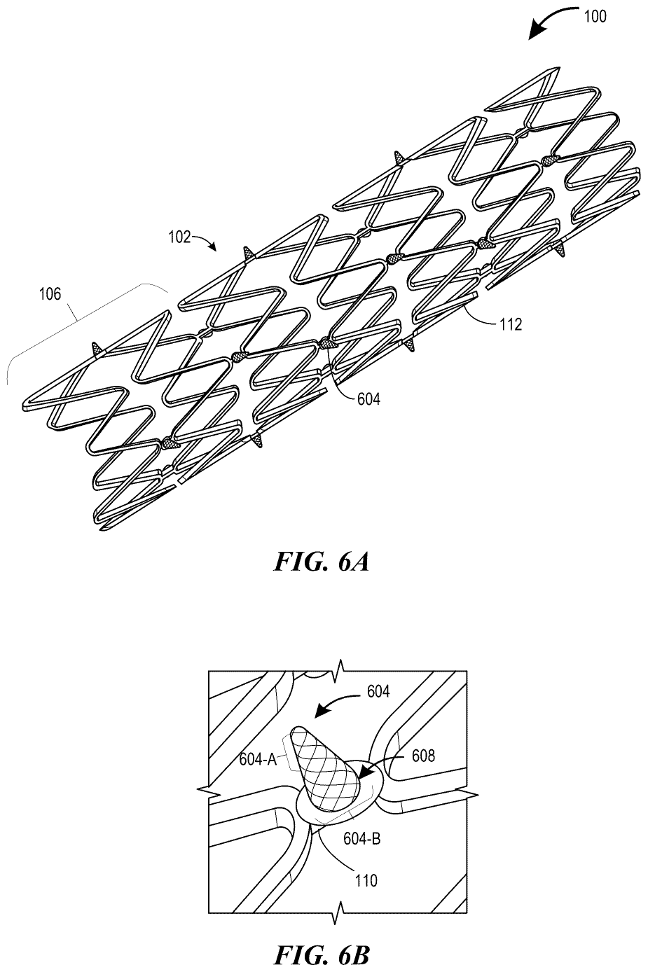

In the embodiments of FIG. 1A, the stent device 100 also includes radiopaque markers 108 (e.g., radiopaque markers 108-1, 108-2, and 108-3 collectively referred to as “radiopaque markers 108”). The radiopaque markers 108 are configured to make certain portions or key features of the stent device 100 visible under fluoroscopy or X-ray enabling physicians to precisely position, deploy, and confirm placement of the stent device. The radiopaque markers 108 can enhance intra-procedural guidance and post-deployment verification without affecting stent function. The radiopaque markers 108 can also be used for informing of the status of stent resorption through noninvasive imaging. This can be achieved based on, for example, radiopaque markers 108 having features that dissolve or change shape as the stent device 100 dissolves. The radiopaque markers 108 can include radiopaque materials, such as gold, tungsten, tantalum, platinum, barium, bismuth, amalgam, iridium (Ir), silver (Ag), barium sulfate (BaSO₄), bismuth subcarbonate (Bi₂O₂(CO₃)), bismuth oxide (Bi₂O₃), tantalum oxide (Ta₂O₅) and zirconium dioxide (ZrO₂) and/or any combinations thereof. In FIG. 1A, the radiopaque markers 108 have a circular shape. The radiopaque markers 108 can be configured in a variety of shapes and sizes (as described with respect to FIGS. 6A-6B).

The radiopaque markers 108 can be positioned along the surfaces of the struts 112 of the tubular frame 102. In some embodiments, the radiopaque markers 108 can be positioned at the opposing end regions and/or at a medial region of the stent device 100. For example, the tubular frame 102 has a first end portion proximate the first open end 102-1, a second end portion proximate the second open end 102-2, and a medial portion between the first and second end portions. The radiopaque marker 108-1 is positioned at the first end portion, the radiopaque marker 108-2 is positioned at the second end portion, and the radiopaque marker 108-3 is positioned at the medial portion between the first and the second end portions. The radiopaque markers 108-1, 108-2, and 108-3 can be radially offset from each other. For example, the radiopaque markers 108-1, 108-2, and 108-3 are positioned at different angular locations around the circumference of the tubular frame 102 rather than all aligned at the same “clocking” angle. In other words, looking at a cross-section, the markers are offset by some radial (angular) spacing around the tubular frame's surface. In some embodiments the markers can take the form of an elongated element (e.g., a band) that extends around a partial or fully-circumferential portion of the stent frame 102. In some embodiments, the markers can be positioned on other portions of the stent frame, such as on apices, along the struts, and/or on links. In some embodiments the markers can be embedded in the stent frame 102 or positioned on the surface of the stent frame 102. In some embodiments, the markers can be positioned on an inner surface of the tubular frame 102 facing the interior of the vessel lumen, or the markers may be on an exterior surface of the stent frame 102 facing toward the adjacent inner vessel wall. In some embodiments, the markers may be coated with or otherwise at least partially encapsulated in an insulative and/or other type of coating that can, for example, reduce or prevent galvanic corrosion.

FIG. 1B illustrates the stent device 100 in the unexpanded state (e.g., also a nominal state or a delivery state) and FIG. 1C illustrates the stent device 100 in an expanded state (e.g., also an exploded state or a stretched state). At the unexpanded state in FIG. 1B, the stent device 100 has a cross-sectional diameter D1 and in the expanded state the stent device 100 has a cross-sectional diameter D2 which is greater than D1. In some embodiments, the expansion ratio D2 to D1 can be greater than six (e.g., greater than two, greater than three, greater than four, greater than five or greater than six). The diameter D2 can correspond to the maximum diameter the stent device 100 is capable of achieving based on the stent's nominal diameter (D1), the strut properties (e.g., cross-sectional dimensions of the struts and strut pattern), and properties of the materials and/or coatings of the struts. When positioned in situ inside a vein, the expansion of the stent device 100 can be partially limited by the vein diameter, compliance, and the presence of other factors, such as thrombus, fibrosis, or external compression. Also, the method of deployment (e.g., balloon expansion vs. self-expansion) can affect the stent's expansion when deployed.

In some embodiments, the diameter D1 is below 3 millimeters, below 4 millimeters, below 5 millimeters, below 10 millimeters, or below 20 millimeters. For example, the diameter D1 can range from 3 to 5 millimeters, from 4 to 10 millimeters, or from 4 to 20 millimeters. In some embodiments, the stent device 100 can be compatible with a stent delivery system that includes delivery through a catheter or sheath having an outer diameter ranging from 8 to 12 French (Fr), from 9 to 11 Fr, or from 12 to 14 Fr. The diameter D2 of the stent device 100 in the expanded state can range from 5 millimeters to 10 millimeters, from 5 millimeters to 20 millimeters, from 10 millimeters to 20 millimeters, or from 10 millimeters to 25 millimeters. As an example, the diameter D1 at the unexpanded state is below 4 millimeters (e.g., 11 Fr delivery profile) and the diameter D2 in the expanded state is at least 10 millimeters. As another example, the diameter D1 can be 2 mm (e.g., 6 Fr delivery profile) and diameter D2 is 13 mm (e.g., an expansion ratio of greater than six). As yet another example, the diameter D1 can be 3.3 mm (e.g., 10 Fr delivery profile) and diameter D2 is 20 mm (e.g., applicable for iliofemoral venous applications).

In some embodiments, the stent device 100 has a length (e.g., a distance between the end regions of the stent device 100 along the longitudinal axis) that can range from 20 millimeters to 50 millimeters (short), 60 millimeters to 100 millimeters (short-to-moderate length), or from 100 millimeters to 150 millimeters (long). In some instances, multiple stents can be deployed in tandem to produce even longer stent structures.

FIG. 1D is a cross-sectional view of the stent device 100 taken along the reference line A-A′ of FIG. 1A, in accordance with the present technology. In the embodiment of FIG. 1D, the stent device 100 has a circular cross-sectional shape. Specifically, the struts 112 are arranged in a symmetrical circle around the circumference of the stent device 100. The cross-sectional shape can also be, for example, elliptical, rectangular, or have a free-form shape. Further, as explained above, the stent device 100 is flexible and has a high compression rate. The cross-sectional diameter of the stent device 100 can therefore be compressed, twisted, or squeezed to have different shapes and sizes.

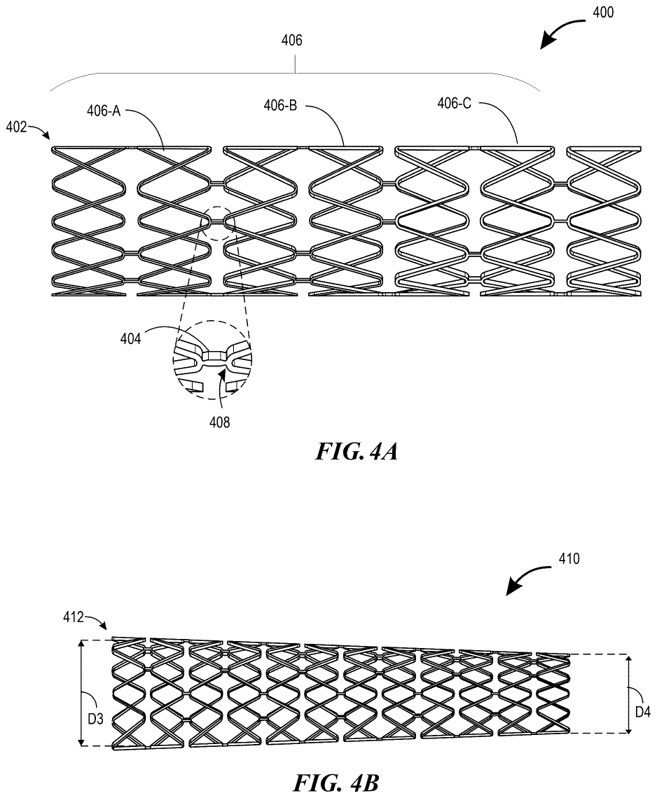

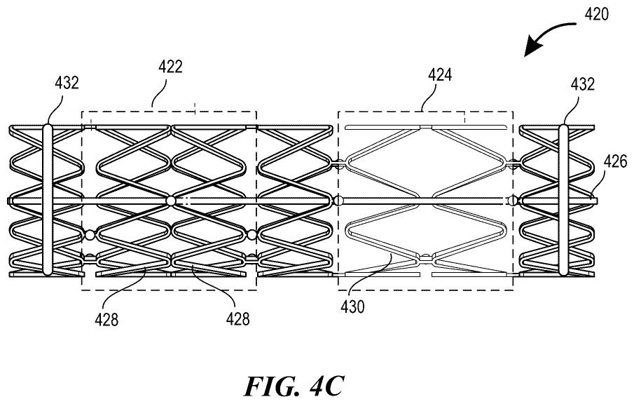

In the embodiment of FIG. 1D, the struts 112 have a square-shaped cross-section. The struts can also have rectangular, round, ellipsoidal, or free-form cross-sectional shape. For example, the strut can have a cross-sectional shape of a flattened circle. The shape and/or dimensions of the strut cross-sections can be tailored to provide desired structural properties. For example, thin struts can make the stent device 100 more elastic while thicker struts provide more support and stiffness. Further, the strut thickness affects the degradation time of the stents. In some embodiments, the cross-sectional dimensions (e.g., a width (W), a thickness (T), or a diameter) can range from 10 micrometers to 50 micrometers, from 75 micrometers to 100 micrometers, from 50 micrometers to 100 micrometers, from 100 micrometers to 250 micrometers, from 125 micrometers to 150 micrometers, from 100 micrometers to 300 micrometers, from 75 micrometers to 450 micrometers or from 100 micrometers to 450 micrometers. A thickness of the strut refers to a distance between a surface S1 facing inwards and a surface S2 facing outwards in the tubular frame. For example, the square-shaped struts 112 shown in FIG. 1D can have a width and a thickness ranging from 75 micrometers to 450 micrometers, from 100 micrometers to 300, or from 125 micrometers to 150 micrometers. In some embodiments, the width of the strut 112 can be greater than the thickness of the strut. For example, the strut can be shaped to be thin but wide so that the ratio between the width of the strut and the thickness of the strut is from 2 to 1 or 10 to 1 (e.g., the strut has a thickness of 25 micrometers and a thickness of 150 micrometers). The thickness and/or the width of the struts can vary along with the length of the stent device 100 (e.g., as described with respect to FIG. 4A).

As explained with respect to FIG. 1A, the strut 112 is made of Mg alloy. In the cross-sectional view, the central portion 120 of the strut cross-section is made of the Mg alloy. The struts can further include one or more coatings 122 (e.g., thin film coatings). In the embodiment of FIG. 1D, the coatings 122 are disposed uniformly across the surfaces of the strut 112. The coatings 122 can also be disposed non-uniformly. For example, a coating can be applied to less than all of the surface of the strut (e.g., on the inner surface of the stent device 100 but not on the outer surface of the stent, or vice versa). A coating can also be disposed only on one or more portions of the stent device 100 (e.g., as described with respect to FIG. 4C). The coatings 122 can be tailored to provide control over a variety of features of the stent device 100. The coatings 122 can, for example, be tailored to control resorption process of the Mg alloy and enhance mechanical properties (e.g., flexibility vs. stiffness) of the stent. The elasticity of the coatings 122 can be controlled by molecular weights and/or viscosity of the coating materials. The coatings described herein can be critical in inflow veins where conventional, non-coated stent devices, have a high likelihood of failing patency.

The coatings 122 can be made of bioresorbable materials. Such materials can include, for example, polylactic acids including PLLA, Poly(D-lactic acid) or poly(D-lactide) (PDLA), Poly(D,L-lactic acid) or poly(D,L-lactide) (PDLLA), polyglycolic acid (polyglycolide) (PGA), poly(lactic-co-glycolic acid) (PLGA), polycaprolactone (PCL), Poly(3-hydroxybutyrate) (PHB), poly(3-hydroxybutyrate-co-3-hydroxyvalerate) (PHBV), poly(sebacic anhydride), poly(adipic anhydride), poly(ortho ester), tyrosine-defined polycarbonates, trimethylene carbonate copolymers, PEG, fluoropolymers including fluorinated ethylene propylene (FEP) or polytetrafluoroethylene (PTFE), or any combinations thereof. As an example, a coating of PTFE as an outer coating can provide lubricity during trackability and promote stronger endothelial response. The combination of PLLA and PTFE, either mixed or as separate coatings, can provide dual benefits: buffering against core material degradation (e.g., of the Mg alloy) while improving trackability and endothelialization. Coatings that can slow down degradation processes include metal oxide, metal hydroxide, inorganic nonmetallic, organic or polymer coating, and their composite coatings. For example, one or more strut coatings can have a different (faster or slower) degradation speed compared to the degradation speed of the Mg alloy.

In some embodiments, the coatings include materials that are non-resorbable. Non-resorbable coatings can provide corrosion resistance, prevent premature degradation, reduce thrombosis by masking the underlying degrading material, or be used for surface modifications. Coatings that are non-resorbable include poly(ethylene-co-vinyl acetate) (PEVA), poly(n-butyl methacrylate) (PBMA), poly(styrene-b-isobutylene-b-styrene) (SIBS), poly(vinylidene fluoride-co-hexafluoropropylene) (PVDF-HFP), poly(vinylidene fluoride) (PVDF), poly(methyl methacrylate) (PMMA), poly(2-hydroxyethyl methacrylate) (PHEMA), polyurethane (PU), polycarbonate urethane (PCU), silicone elastomer coatings, polysulfone (PSU), polyethersulfone (PES), polyether ether ketone (PEEK) thin films, polyimide coatings, epoxy-based thin film coatings, fluorinated ethylene propylene (FEP), polytetrafluoroethylene (PTFE), expanded PTFE (ePTFE) thin surface layers, tetrafluoroethylene-hexafluoropropylene-vinylidene fluoride terpolymers, phosphorylcholine polymer coatings, covalently bound heparin coatings, albumin-binding surface coatings, polyethylene glycol (PEG) grafted surfaces, poly(oligoethylene glycol methacrylate) (POEGMA) brushes, zwitterionic sulfobetaine coatings, carboxybetaine polymer coatings, plasma-polymerized fluorocarbon coatings, plasma-deposited parylene coatings (Parylene C, Parylene N), silicon carbide (SiC), diamond-like carbon (DLC), amorphous carbon coatings, titanium nitride (TiN), titanium nitride oxide (TiNOx), titanium oxynitride, tantalum nitride coatings, chromium nitride coatings, iridium oxide thin films, gold thin film coatings, platinum-iridium surface coatings, hydroxyapatite thin coatings (non-resorbable forms), alumina (Al2O3) thin films, zirconia (ZrO2) coatings, hafnium oxide thin films, graphene coatings, graphene oxide surface layers, carbon nanotube coatings, silane-based self-assembled monolayers, nitric-oxide releasing xerogel coatings (non-degradable matrix forms), silicone-based drug reservoir matrices, polyacrylonitrile (PAN) coatings, and polyvinyl alcohol (PVA) crosslinked non-degradable coatings. Additionally, the one or more coatings 122 can be impregnated with radiopaque materials like iridium or zinc to provide visibility during placement and creating a long-term landmark of the stent location even after complete resorption.

In certain embodiments, the coatings can be non-uniform across the length of the stent device, around the diameter of the stent, and/or around the circumference of an individual strut. For example, the stent frame may have a greater thickness on an outer strut surface in comparison to an inner strut surface. In some embodiments, the stent devices may have different coating types on different areas of the stent frame and/or an individual strut, whether biodegradable and/or non degradable. For example, on a strut, the inner surface and side walls can include a first coating, and the outer surface can have a second coating different than the first coating.

In some embodiments, the coatings 122 include graft materials. A coating of graft material can be disposed on an inner or outer surface of the stent device 100, or encapsulate the stent device 100 fully. The graft materials can provide openings that promote rapid endothelialization while maintaining sufficient material presence to reinforce the stent structure. Graft materials can include bioresorbable polymers or fluoropolymers such as polyester, FEP, PTFE, or a combination thereof. Graft material coatings with engineered openings can enable controlled blood or side-branch flow while maintaining coverage to seal or exclude target lesions. By tailoring hole size, shape, and placement, the graft material coating can balance flow preservation with hemostasis and reduce the risk of branch obstruction.

The coatings 122 can further be impregnated with one or more bioactive therapeutic agents enabling localized therapeutic delivery of therapeutic agents when the stent is deployed. The therapeutic agents can be applied for reducing inflammation, cellular level uniry, thrombosis, etc. For example, coatings made of co-polymers of L-lactide and ε-caprolactone, terpolymers of L-lactide, glycolide and ε-caprolactone, and resorbable stents made from polylactic acid or polyglycolic acid can be used for therapeutic agent-release coatings. The therapeutic agents can be configured to, for example, support vessel healing and remodeling, reducing the risk of re-thrombosis or restenosis and complications after the deployment procedure. The types of therapeutic agent-release coatings may include properties that are antithrombotic (e.g., heparin), anti-inflammatory (e.g., dexamethasone), anti-collagen (e.g., collagenase), pleotropic impact (e.g., inflammatory) aspects of statins, or anti-spasmodic, masking foreign body interactions and/or targeting specific biological pathways (e.g., via statins). In some embodiments, the therapeutic agent can be impregnated to a coating providing a delayed or slow degradation (e.g., chitosan-impregnated Cu(II) coating).

The therapeutic agent coating may exhibit any one or combination of the following properties: anti-thrombotic (e.g. heparin, bivalirudin, fondaparinux, statins) to treat preexisting thrombus and prevent the formation of new thrombus; anti-inflammatory (e.g. Dexamethasone, NSAIDs, Remicade, Orencia, Rituxan, Actemra, icam-1, E-selectin and P-selectin) to arrest the inflammatory process that was pre-existing (before stent placement) or prevent one which may be caused or aggravated by stent implantation; anti-collagen (e.g. collagenase) to breakdown collagen within the lesion that is occluding the vessel and/or mural collagen; anti-spasmodic (e.g. Antimuscarinics, Dicyclomine, Hyoscyamine, Atropine, Scopolamine, Tolterodine, Flavoxate, Oxybutynin, Mirabegron) to allow relaxing of the smooth muscles lining the vessel and prevent vessel spasms which may occur as a result of mechanical stimuli from stenting or other interventions; anti-platelet (e.g. Clopidogrel aka Plavix, ASA, Aspirin, Asaphen, Entrophen, Novasen, prasugrel, ticagrelor, warfarin); Anti-proliferative (e.g. 5-fluorouracil, cisplatin, doxorubicin, Imatinib, rituximab, trastuzumab, Paclitaxel, sirolimus, Azathioprine, cyclosporine, Zotarolimus) to suppress the immune system's response to stenting and reduce local inflammation; of properties for masking foreign body interactions and/or targeting specific biological pathways (e.g. via statins). An example of targeting specific pathways is helping to enhance endothelialization or smooth muscle proliferation, which can ultimately embed the stent within the vessel wall. Another example includes therapeutic agents that are used in the treatment of venous disease, such as warfarin, Eliquis, and/or any Vitamin K antagonists. The therapeutic agent may be chemical, biological, and/or biosimilar in nature. The thickness of the therapeutic agent coating may be specific to how much therapeutic agent is needed to be delivered (for example, chronic lesions may require more therapeutic agent to be delivered), while also potentially contributing to delaying the time for resorption of the actual Mg alloy material. Another variation is that this therapeutic agent is blended with the alloy of the stent material, in lieu of a coating. This would be advantageous for treatments where the therapeutic agent release over a longer period of time is desirable, i.e., lower therapeutic agent elution rate.

As an example, a stent can include multiple coatings with varying materials and properties. The multiple coatings can include an outermost layer of a first coating, a second outermost layer of a second coating, a third outermost layer of the first coating, and a fourth outermost layer of the second coating. The first coating can be a bioactive therapeutic agent coating, as previously described. A second coating can provide delay of stent degradation. Such configuration can allow for initial vessel healing by therapeutic agent release from the first outermost layer, followed by a period that the stent is performing its mechanical function without degradation and a subsequent release of another dose of bioactive therapeutic agent coating from the third outermost layer of the first coating, followed by a period that the stent provides purely its mechanical function (via the second coating), before the stent begins to resorb. In some embodiments, the coatings (e.g., an outermost coating of a strut) can be textured to mimic biological structures (e.g., nano-textured, micro-textured, biomimetic flow surface, surface gradients).

In some embodiments, the structural properties and/or applied coatings can be used to reduce thrombosis risk when the stent device is deployed in a vein. For example, experimental studies show that a stent device with less thrombogenic materials and thinner strut thicknesses can reduce thrombosis. A stent device with 150 micrometer strut thickness made of bare nickel-titanium alloy produced 0.073 grams of thrombus; a stent device with 150 micrometer strut thickness made of Mg alloy with 5-10 micrometer PLLA coating produced 0.053 grams of thrombus; and a stent device with 457 micrometer strut thickness made of Mg alloy with 5-10 micrometer PLLA coating produced 0.087 g of thrombus. The experiments were carried out as a blood loop for a few hours and measuring the thrombus weigh by scaling. In another animal study run for 19 days, endothelialization rates of the stents were measured (as the percentage of the stent device that was covered by new tissue growth) and it was found that stents with PLLA had greater new tissue growth encapsulating the stents than a bare metal nitinol (NiTi) stent.

The stent device 100 can be used for opening and/or maintaining the liminal patency of obstructed and/or otherwise compromised (e.g., collapsed or enlarged) vessels, hollow bodies, and/or other lumens in the body. Such obstruction or weakening of vessels (e.g., aneurism, varicosed veins) can be caused, for example, by cancer, trauma, inflammation, motion, scarring, atherosclerosis, hypertension, and/or other cardiovascular conditions. The stent device 100 can also be applied for relining previously implanted stents. The stent device can be used in vessels including, for example, the femoral, tibial, popliteal, fibular, saphenous, or femoral veins (e.g., profunda femoris) (e.g., vessels at inguinal ligament, adductor canal, or knee).

The stent device 100 can be applied to treat conditions of the vascular system, such as VTE, PTS and NIVL. VTE encompasses blood clots in the venous system, primarily deep vein thrombosis (DVT), and pulmonary embolism, and is a major cause of morbidity and mortality driven by stasis, endothelial injury, and hypercoagulability. DVT refers to thrombus formation in deep veins-most commonly in the lower extremities-causing pain, swelling, and risk of embolization to the lungs; diagnosis typically relies on duplex ultrasound, and treatment focuses on anticoagulation with selected interventions to restore patency. PTS is a chronic complication following DVT characterized by persistent limb pain, edema, skin changes, and in severe cases venous ulceration due to sustained venous hypertension, impairing quality of life and mitigated by optimal DVT management and compression therapy (e.g., preventing chronic and acute obstruction and occlusion of veins and preventing impaired drainage caused by valve damage/reflux). NIVL encompasses extrinsic iliac vein compression (e.g., May-Thurner anatomy) or intrinsic non-thrombotic webbing/spurs stenosis that restricts venous outflow and can mimic or contribute to DVT-like symptoms; endovascular evaluation and stenting are commonly used to restore lumen and improve venous return. The stent device can also be applied for opening of vessels in the upper extremity veins, inferior and superior vena cava, pelvic veins, colon, rectum, esophagus, ureters, lymphatic vessels and nodes, sinus, cerebral and neck veins, etc. Other related applications include placement in patients with arteriovenous fistulas, as maintaining patency is a prevalent and known challenge and veins where pacing leads are or have been introduced, as these veins become prone to injury and scarring, resulting in patency issues. Further, the stent device can be applied to tubular, hollow structures in the body beyond vessels, such as urinary bladder, urethra, uterus, fallopian tubes, etc.

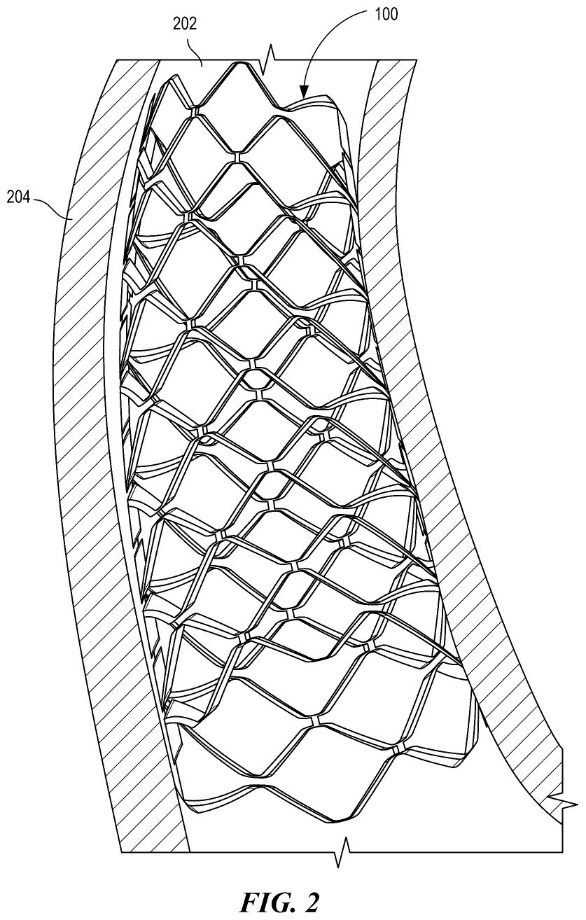

FIG. 2 is an isometric view of the stent device 100 of FIGS. 1A-1D deployed within a lumen 204 (e.g., a vein, an artery, or other anatomical lumen) in accordance with some embodiments of the present technology. As shown in FIG. 2, the stent device 100 can be deployed intraluminally (e.g., inside the vein) and expanded so all or a portion of its struts press circumferentially against an internal surface 202 of the wall of the lumen 204 to restore and maintain lumen patency. Proper sizing and placement of the stent device 100 is configured to ensure a uniform radial apposition to the vein's wall. The stent device 100 is configured to have a length and positioning to span the stent device 100 across compressed or scarred segments of the lumen 204 so that the stent device 100 resists external compression and remains stable in its position. Further, anchoring protrusions (e.g., the anchoring protrusions 104 in FIG. 1A) extending away from the outer surface of the stent device 100 can penetrate the internal surface 202 of the lumen 204 to prevent migration of the stent device 100.

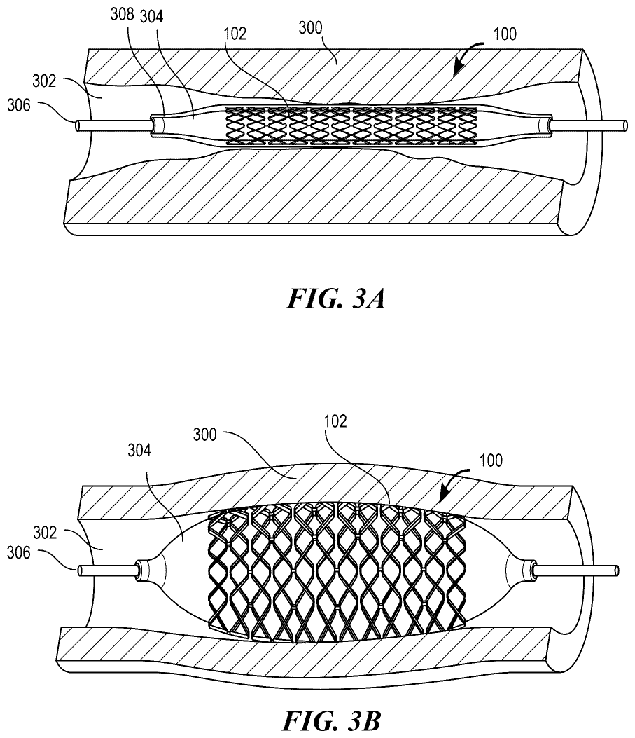

FIGS. 3A and 3B illustrate the stent device 100 at different stages of deployment within a lumen 300 (e.g., a vein) in accordance with some embodiments of the present technology. Specifically, in FIG. 3A, the stent device 100 is shown inside the lumen 300 after a delivery (e.g., the stent device 100 is in an unexpanded state) and in FIG. 3B, the stent device is shown inside a vein after being expanded (e.g., the stent device 100 is in an expanded state). In FIG. 3A, the stent device 100 is coupled with a delivery catheter 306 including a balloon catheter 304. Specifically, the stent device 100 is coupled with the balloon catheter 304 so that the balloon catheter 304 is at least partially positioned in the lumen of the stent device 100. The coupling can be achieved by a sheath 308 at least partially surrounding the stent device. The sheath 308 is a thin, tubular, or other cover that constrains the stent device 100 and the delivery catheter 306 while they are pushed through the vein during delivery. The stent device 100 is thereby positioned between the balloon catheter 304 and the sheath 308. The delivery catheter 306 including the balloon catheter 304 and the stent device 100 can be guided to the lumen 300 with a guidance sheet (not shown) that facilitates delivery of the delivery catheter 306 to a target site.

The balloon catheter 304 can be made of nylon, polyester, silicone, urethane, thermoplastic elastomers (e.g., Pebax), PET, polyurethane, polyethylene, polyolefin, polyvinyl, or a combination or blend thereof. The balloon catheter 304 can be semi-compliant or compliant. The balloon catheter 304 is configured to be inflated, as shown in FIG. 3B, so that the balloon catheter 304 causes the stent device 100 to be deformed. The balloon catheter 304 forces the stent device 100 to press against the inner wall 302 of the vein so that the vein lumen defined by the inner wall 302 is expanded as well. In some instances the balloon catheter 304 can expand the stent device 100 to be oversize compared to the lumen of the lumen 300. The oversizing can resist migration by creating frictional resistance. Further, the spaces between the struts can engage with the tissue of the lumen 300 and pinch the tissue for fixation. Once the stent device 100 is positioned at the target site and expanded, the balloon catheter 304 can further be deflated and the delivery catheter 306 with the balloon catheter 304 can be removed from the vein. The stent device 100 remains inside the vein to support blood flow through the vein. In some embodiments, the stent device 100 can be self-expandable. A self-expandable stent can include shape-memory or superelastic materials that automatically expand to a predetermined diameter once deployed in a vessel. The use of the stent device 100 is described here with respect to applications for treatment of cardiovascular conditions affecting veins. However, in some embodiments, the stent device 100 can also be used for treatment of arterial conditions in a similar manner.

In some embodiments, a balloon catheter 304 includes two or more balloon sections positioned in tandem or sequentially along the longitudinal axis of the balloon catheter. For example, the balloon catheter 304 can include a first balloon section and a second balloon section that are independently inflated and therefore independently control the expansion of separate sections of the stent device 100. For example, the first balloon section can be used to expand a first portion of the stent device 100 to a first expanded diameter and, subsequently and/or simultaneously, the second balloon section can be used to expand a second portion of the stent device 100 to a second expanded diameter. The first expanded diameter and the second expanded diameter can be different from each other and/or the same. In this manner, the multiple balloon delivery can provide for staged delivery of the device and/or customized sizing of portions of the stent device 100.

FIGS. 4A-4C illustrate various stent devices configured in accordance with some embodiments of the present technology. The embodiments illustrated in FIGS. 4A-4C can include some or all of features of the stent device 100 and can be used in a similar manner as the stent device 100. Specifically, these embodiments can be designed to have certain features that address particular challenges of stent veins.

FIG. 4A is a side view of a stent device 400 with a varying strut thickness, in accordance with the present technology. The stent device 400 includes a tubular structure 402 with multiple strut patterns 406 (e.g., strut patterns 406-A, 406-B, and 406-C) coupled adjacent to each other via connectors 404. The strut patterns 406 can correspond to the strut patterns 106 described with respect to FIG. 1A, with the exception that the strut patterns 406 include struts with varying thicknesses (e.g., having a larger cross-sectional diameter in one or more directions). In FIG. 4A, the struts of the strut pattern 406-C have a greater thickness than the struts of the strut pattern 406-B, and the struts of the strut pattern 406-B have a greater thickness than the struts of the strut pattern 406-A. In FIG. 4A, the thickness is shown as increasing radially from the strut pattern 406-A positioned at a first end region of the stent device 400 toward the strut pattern 406-C positioned closer to a second, opposing end region of the 400. The stent device 400 with varying thickness can provide varying flexibility across the length of the stent. The stent device 400 can be used in instances where a portion of the stent device 400 may need to sustain motions from joint movement (e.g., the strut pattern 406-A), such as being placed across the inguinal ligament, while other sections (e.g., the strut pattern 406-B) extend outside of the joint and need greater stiffness. The varying stiffnesses can be achieved by varying strut thicknesses as greater thickness increases the stiffness of the stent structure.

In some embodiments, the strut thicknesses can have localized variations, as shown in the exploded view of the connector 404 in FIG. 4A. As illustrated, the thickness of the strut of the connector at a location 408 adjacent to the intersect of the struts (e.g., a location where a peak region of a strut pattern is connected to the connector 404) is reduced to provide increased flexibility to the intersect. In some embodiments, a thickness can be increased at a particular location of a strut to change the stiffness.

FIG. 4B is a side view of a stent device 410 with a tapered (or conical) structure, in accordance with the present technology. As illustrated in FIG. 4B, the tubular structure 412 of the stent device 410 has a gradually decreasing diameter so that a first end of the stent device 410 has a diameter D3 and a second, opposing end of the stent device 410 has a diameter D4 where D3 is greater than D4. The stent device 410 having such a tapered configuration can be applied to a vein having a varying diameter. In FIG. 4B, the diameter is tapered throughout the length of the stent device 410. In some embodiments, only a portion of the stent (e.g., an end region) of the stent is tapered. Also, in some embodiments, the diameter of the stent can vary along the length of the stent in a non-gradual manner. For example, the stent can include multiple sequential portions with diameters varying between the diameters D3 and D4. As another example, a stent can include sequential portions having varied, non-tapered diameters. The varying strut thickness illustrated in the stent device 400 and the varying diameter of the stent device 410, illustrated in FIGS. 4A and 4B respectively, can be used to achieve varying levels of radial force along the length of the stent for situations where the stent may originate from a larger vessel and extend into a smaller one, e.g., femoral to tibial.

FIG. 4C is a side view of a stent device 420 with a longitudinal supporting spine 426 and varying structure, in accordance with the present technology. As illustrated in FIG. 4C, the supporting spine 426 extends along the length of the stent device 420 (e.g., along a central axis of the stent device 420). The spine 426 is positioned between adjacent struts and extend in a longitudinal direction at least partially between a first end region and an opposing second end region of the stent device. The supporting spine 426 is configured to provide structural support to enhance the stent's cohesive integrity while maintaining flexibility. The supporting spine 426 can have a same cross-sectional diameter as the struts (e.g., the struts 428 or 430) of the stent device 420. For example, the supporting spine 426 can be made of the same strut as the struts of the stent device 420. The supporting spine 426 can also have a different cross-sectional diameter. For example, the supporting spine 426 can have a greater cross-sectional diameter than the struts of the stent device 420 for additional stiffness and structural support.

The supporting spine 426 can extend throughout the full length of the stent device 420 (e.g., from a first end region to a second, opposing end region), as shown in FIG. 4C. In some embodiments, the supporting spine 426 extends throughout a portion of the length of the stent. A partially positioned supporting spine can be positioned in the middle region of the stent or be extending from an end region of the stent. In some embodiments, the supporting spine 426 is made of the same bioresorbable material as the stent itself (e.g., as described with respect to FIGS. 1A and 1D). Alternatively, the supporting stent can be made of a different material or include different coatings than the stent. The supporting spine can have various cross-sectional profiles (e.g., circular, elliptical, rectangular, or free-shape). In some embodiments, the stent device 420 is coupled with multiple supporting spines 426 (e.g., two, three, or four) that are positioned along the circumference of the stent device 420. For example, the stent device 420 can include two supporting spines positioned at 180 degrees along the circumference of the stent device 420. In some embodiments, the supporting spine 426 extends along the length of the stent device 420 in a spiral manner.

The supporting spine 426 can be coupled or attached to the stent device 420 using various coupling mechanisms, including biocompatible adhesives formulated for bioresorbable materials; crimping mechanisms using pieces of the bioresorbable material (e.g., struts) looped around attachment points on the struts of the stent device 420; weaving mechanisms using the supporting spine 426 through the strut patterns of the stent device 420; thermal bonding mechanisms; or mechanical interlocking mechanisms. In some embodiments, radiopaque markers (e.g., the radiopaque markers 108 in FIG. 1A) can be used to connect the supporting spine 426 to the stent device 420. The supporting spine 426 can be positioned on the outer surface or the inner surface of the stent device 420 or both. In some embodiments the spine 426 and/or portions thereof can be made of a radiopaque material and can be used in conjunction with a markers (e.g., marker bands) or in place of the markers. For example, in some embodiments, stent devices can include multiple spines 426 that extend along at least a portion of the length of the tubular frame to provide proper visualization of the length of the stent frame as well as its expanded lumen profile (e.g., diameter).

FIG. 4C further illustrates sections with varying structures. In addition to stent diameter or strut thickness, the structure of the stent can be used to achieve varying levels of radial force along the length of the stent, which is needed for situations where the stent extends from a larger vein into a smaller vein, e.g., femoral to tibial. In FIG. 4C, the stent device 420 includes a section 422 having a first structure and a section 424 having a second structure. The structure of the section 422 can be described as “closed” in that all the struts 428 along the circumference of the stent device 420 are connected to it from their apices. In contrast, the structure of the section 424 can be described as “open” in that only a portion of the struts 430 are connected from their apices. The closed structure of the section 422 provides stiffness whereas the open structure of the section 424 provides more flexibility. Further, the struts 430 of the section 424 are longer (e.g., a distance between opposing peak regions of the strut pattern is greater) compared to the struts 428 in the section 422. The longer struts can further provide additional flexibility. Further, the strut patterns can include one or more openings of variable shapes to provide localized increased flexibility. In some embodiments, stent frames can have struts with struts widths that vary across the length of a strut, strut thicknesses that vary across a strut's length (i.e., non-uniform thicknesses and widths). In some embodiments, stent frames can have non-uniform thicknesses and widths of the apices and links (e.g., the width and/or thickness of an individual apex and/or link can change across its length). In some embodiments, stent frames can have non-straight structures, in which links and/or struts are arched, wavy, curled, zigzag, and/or in a other complex shapes. In some embodiments, stent frames can have vary the position of links, connecting strut to strut, or apex to strut, or apex to apex. These variations can be used to affect mechanical performance of the expanded stent, expansion properties of the stent, flexibility in delivery, and/or stability of the stent on the balloon catheter during delivery.

Also, other structural and/or material features of the stents can be varied along different sections of the stent. For example, a stent can include sections made of dissimilar metals, compared to the Mg alloy described with respect to FIG. 1A, to induce a surface charge to promote vasodilation and induce positive remodeling, while reducing risk of galvanic corrosion. As another example, a stent can include sections that resorb non-uniformly. Non-uniform resorption can be used in instances where the stent is positioned to cross an ostium or pass across a bifurcation where faster resorption is desired. As an example, in patients with lesions requiring long-term support, certain portions of a stent device can incorporate non-resorbing or slower-resorbing materials, achieved through thicker coatings and/or materials with varying resorption times (or being completely non-resorbing). The non-uniform resorption can be achieved by modulating the thickness of the Mg alloy material (e.g., as described with respect to FIG. 4A) and/or adding different coatings to sections where delayed resorption or non-resorption is desired. Also, strut structures having a greater surface area (e.g., the open structure of the section 424) can be resorbed at a greater pace due to an increased exposure to blood. Additional structural features can be added, such as adding holes to the struts, in locations where faster resorption is desired. Further, multiple stents devices can be used in tandem, at least partially overlapping, or as branched. For example, a shorter non-resorbing stent device can be placed within a longer resorbing stent device.

In the embodiment of FIG. 4C, the stent device 420 further includes constraining features 432 (e.g., a ring). The constraining features can be added to ensure that certain portions (e.g., the end portions of the stent device 420) of the stent device 420 do not expand beyond a desired diameter. The constraining features can be configured as rings, loops, or ribs extending at least partially around the circumference of the stent device 420. The constraining features 432 can be made of the bioresorbable materials described, for example, with respect to FIGS. 1A and 1D. The constraining features 432 can facilitate repositioning of the stent device 420 during delivery (e.g., as shown in FIGS. 3A-3B) by providing a smoother pulling and pushing of the stent device with a delivery catheter inside a vein. For example, the strength of constraining features 432 can be tailored to sustain between 20-70% of a total radial force needed for full expansion of a stent device. Once the maximum strength of the constraining features 432 has been exceeded, they are designed to intentionally break. This maximum strength can be “programmed” by the selection of the material, its thickness and/or creating intentional weak points in constraining features 432 that are designed to break when that threshold has been exceeded e.g., via a kink or notch in the rib. The constraining features 432 allow the physician to apply limited pressure to partially expand the balloon catheter (e.g., the balloon catheter 304 in FIGS. 3A-3B) while keeping the end portions of the stent device constrained by the constraining feature to allow repositioning of the stent device in the vein. Once the decision has been made to continue to full stent deployment, the physician can apply full pressure to the balloon catheter, which will break the constraining features 432 and result in the ends of the stent to also expand. In some embodiments, the constraining features 432 can be used to control the expansions of the stent device 420 at a desired positions (e.g., a particular position requiring a narrower diameter). In some embodiments, the constraining features 432 can include radiopaque materials so that the constraining features operate as radiopaque markers (e.g., as discussed with respect to the radiopaque markers 108 in FIG. 1A). The constraining features 432 can also provide resistance for stent device migration by “burying” themselves into the vessel wall.

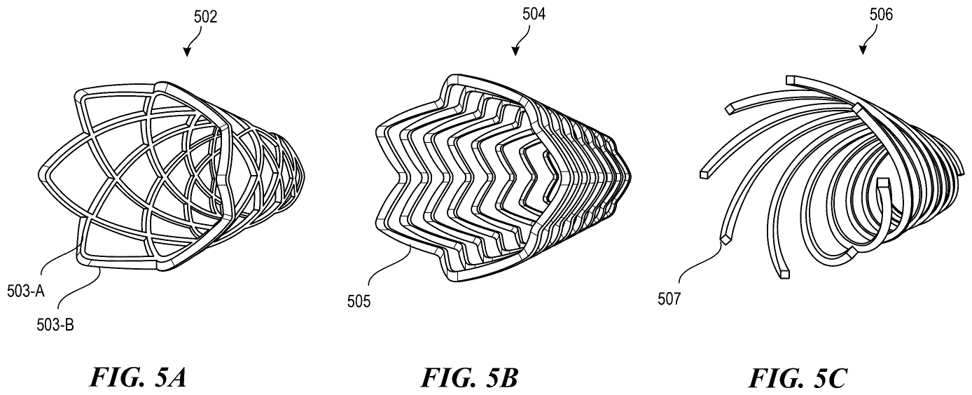

FIGS. 5A-5C are isometric views of stent devices having varying structures in accordance with some embodiments of the present technology. FIG. 5A illustrates a stent device 502 having multiple helical struts having opposing spin directions (e.g., struts 503-A and 503-B) intertwined to form a tubular structure forming the stent. The stent device 502 can have some varying mechanical properties compared to the stent device 100 described with respect to FIG. 1A. For example, the stent device 502 can have different stiffness and/or compression ability compared to the stent device 100.

FIG. 5B illustrates a stent device 504 having multiple ring-shaped struts 505 interconnected by flexible connectors (not shown). The structure with the ring-shaped struts can provide chronic radial force as well as flexibility. For example, the ring-shaped struts 505 have sufficient stiffness to provide radial force, but the stent device 504 has flexibility to easily bend because of the flexible interconnecting members or linkages between the stent rings.

FIG. 5C illustrates a stent device 506 having a helical configuration. As illustrated, the stent device 506 includes multiple struts 507 twisted along the length of the stent device 506 in a helical manner. The helical configuration of the stent device 506 can reduce the structural resistance to bending as well as enhance the resistance to kinking of the stent during bending. In addition to the illustrated strut configurations, the struts can include any other type of patterns including rounded and/or sharp shapes, mesh configurations, net configurations, uniform, or non-uniform patterns, etc.

FIG. 6A is an isometric view of the stent device 100 of FIGS. 1A-1D with radiopaque markers 604 (also referred to as “engagement markers” or “engagement features”) configured in accordance with embodiments of the present technology, and FIG. 6B is an enlarged isometric view of a portion of the stent device 100 of FIG. 6A. In the embodiments of FIGS. 6A-6B, the radiopaque markers 604 correspond to the radiopaque markers 108 described with respect to FIG. 1A, but have a shape configured to engage adjacent tissue (e.g., an inner wall of a vein). For example, in the illustrated embodiment, the markers 604 have conical or dome-like shape with a base portion 604-B and a tip portion 604-A. The tip portion 604-A extends outwards from the base portion 604-B and has a rounded tip-shape. The conical radiopaque markers 604 are positioned within an eyelet 608 (e.g., an opening, aperture, or a partial opening) defined within the outer surface of the tubular frame 102 so that the base portion 604-B is at least partially embedded within the eyelet 608 and the tip portion of the respective marker extends away from the outer surface of the tubular-shaped stent structure. As shown in FIG. 6B, the eyelet 608 can be formed in a connector region 110 that connects adjacent strut patterns 106. Alternatively or additionally, such eyelets can be defined within the struts of the tubular frame 102. The tip portion 604-A of the respective marker can be configured to engage with the inner wall of a vessel to resist stent migration. The radiopaque markers can thereby operate as indicators used in imaging as well as to prevent or resist migration of the stent device 100 after deployment. In some embodiments, the markers 604 may have other shapes suitable for penetrating, grasping, or otherwise engaging adjacent tissue, such as a pointed tip, a hook, a barb, or the like. In some embodiments, the markers 604 do not include radiopaque material and are provided solely for anchoring and/or anti-migration purposes.

In some embodiments, a radiopaque material can be blended with the Mg alloy or be added as a radiopaque coating or ink on the exterior surface of the struts in specific locations or covering the entire tubular frame. The radiopaque markers can be configured in a variety of 2-dimensional or 3-dimensional shapes, such as lines, rectangular, circular shapes, loops, rings, ribs, etc. The thickness of the radiopaque markers and markings can be adjusted based on the desired level of radiopacity. The patterns of placement can take various forms, such as in the form of circumferential rings at a variety of positions on the tubular frame, at a variety of axial orientation across the tubular frame length, and be configured as helical patterns, zigzag patterns, or combinations thereof. An example of a radiopaque marker that changes shape as the stent device dissolves is a ring around the tubular frame that resembles an “O” when visualized cross sectionally and changes to an oval as the stent device dissolves. In some embodiments, the radiopaque markers can be positioned to form a 2-D shape to allow for orthogonal fluoroscopic alignment and placement under radiographic imaging, e.g. “O”, “C”, “L”, “8”, “E”, “T”, “Y”, “U”, “S” shapes, and/or a pattern of markers/markings around a perimeter of the stent device.

FIGS. 7A and 7B are isometric views of strut configurations for stent devices configured in accordance with some embodiments of the present technology. Varying strut patterns can be used to provide different structural properties for the stent device. For example, different strut patterns can be designed to redistribute stiffness and stress, which changes a stent's radial force, flexibility, and crush recoverability. Specifically, FIGS. 7A and 7B illustrate alternative embodiments for the apices of the strut patterns, which in FIG. 1A are shown with rounded V-shaped apices (e.g., the peak region 112-A formed of the strut 112) that define a zig-zag pattern. FIG. 7A is an enlarged isometric view of a portion of a stent device a peak region 700 having a triangular shape in accordance with embodiments of the present technology. In the strut structure shown in FIG. 7A, two adjacent struts 702 are joined by the triangle-shaped peak region 700, with the adjacent struts 702 extending in a generally parallel manner on either side of the peak region 700 when the strut structure when in an unexpanded state. As the stent expands, the parallel struts 702 are drawn outwardly away from each other while the triangle-shaped peak region 700 maintains the structural integrity at the peak region.

FIG. 7B is an enlarged isometric view of a strut structure with a peak region 710 having a hexagonal shape in accordance with the present technology. In the embodiment of FIG. 7B, adjacent struts 712 extend in a generally parallel manner when the strut structure is in an unexpanded state and are joined by the hexagonally-shaped peak region 710 (e.g., the peak region having an at least partially hexagonal shape).

The strut structure shown in FIGS. 7A and 7B both have a lower overall radius of curvature than V-shaped peak regions. This configuration distributes the stress applied to the peak region (i.e., stress caused by expansion and/or contraction during, e.g., delivery loading, deployment, and/or vascular movement) across a greater area than V-shaped peak regions, where the stress concentrates at a single apex. Therefore, a peak region with a lower radius of curvature provides for decreased stress concentrations. When the stent device is deployed in a vein, this can be of particular use as the veins undergo significant expansion and compression (much more so than arteries), and the reduced stress profile can enhance the durability of the stent device. Furthermore, peak regions with reduced curvature require are more difficult to plastically deform, and therefore stent frames having these configurations of peak regions can be more difficult to plastically deform than V-shaped peak regions. This allows the stent device to undergo a higher degree of elastic deformation of the stent, which can be of particular use when in a lumen, such as a vein, which expands and contracts substantially and frequently. In some embodiments, the peak regions can have different polygonal shapes (e.g., rectangular, pentagonal, octagonal, or free-form polygonal shapes). In some embodiments, the peak regions can have round shapes (e.g., circular, ellipsoidal, egg-shaped, or free-form rounded shapes). For example, the struts can form a bulb (e.g., resembling a light bulb) shaped peak region (e.g., the struts 712 form a rounded bulb peak region connecting the parallel struts 712). In some embodiments, a tubular frame of a stent device may have two or more differently shaped peak regions to provide desired structural properties along select regions of the stent device. For example, a central region of a stent frame may include peak regions having a conventional V-shape and end sections may have peak regions with lower radii of curvature to distribute the stress applied thereto across a greater area. In some embodiments, adjacent peak regions may have differing configurations (e.g., an alternating pattern).

In some embodiments, the apices of the strut patterns positioned at the end regions of the stent device can be splayed inwards or outwards when the stent device is in an expanded state. For example, the peak regions may be splayed outwardly (e.g., flare radially outward) at one or both of the end regions to engage adjacent tissue and provide anchoring and anti-migration properties. In some embodiments, peak regions of medial portions of a stent device can be splayed outwardly to provide further tissue engagement.

The strut patterns can be tailored to provide certain structural features. For example, hexagonal strut patterns can distribute loads evenly and enable high flexibility with good crush recoverability and kink resistance, supporting conformability in tortuous segments, while bulb-shaped (enlarged node) patterns tend to increase local radial strength and anchoring at connection points, improving anti-migration and resistance to focal compression but with somewhat reduced overall flexibility and potentially higher foreshortening. The different geometries can be tailored and tuned to trade off radial force, flexibility, fatigue life, radiopacity, and side-branch preservation.

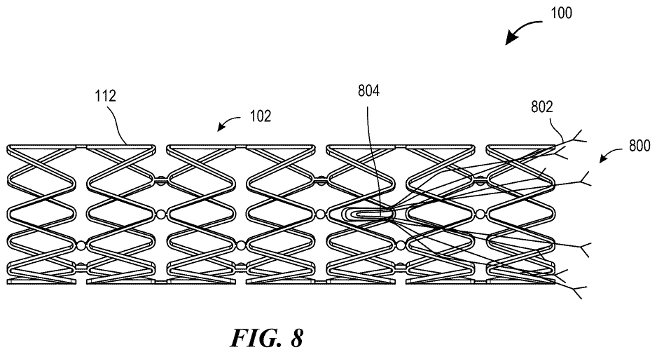

FIG. 8 is a side view of the stent device 100 with a filter 800, in accordance with some embodiments of the present technology. As illustrated in FIG. 8, the filter 800 is positioned at least partially within the lumen of the stent device 100. The filter 800 can be coupled to the tubular frame 102. The filter 800, when positioned with the stent inside a vein in situ, is configured to trap emboli (e.g., masses of undissolved material traveling in the veins, such as blood clots, fat globules, bubbles, or tumor cells). The filter can operate to prevent downstream clinical sequelae caused by emboli.

The filter 800 can be made of the same Mg alloy materials and as discussed with respect to the materials used to make the stent device 100, as described, for example, with respect to FIGS. 1A and 1D. The filter 800 can be configured as being a permanent part of the stent device 100 and being physically attached to the stent device 100. Alternatively, the filter 800 can be configured to be separate from the stent device 100 and be placed within the stent lumen, or distal or proximal to it, when the stent device 100 is placed inside a vein.

In the embodiment illustrated in FIG. 8, the filter 800 (e.g., an inferior vena cava type filter) includes a central hub 804 and multiple legs 802 that extend and expand from the central hub 804. The legs 802 are configured to anchor the filter 800 against the inner surface of the vein tissue and/or against the struts 112 of the stent device 100. The legs 802 form a conical or cage-like structure that can trap the emboli and/or other materials while allowing the blood flow through. Exemplary alternative embodiments of the filter 800 include a basket-type configuration (e.g., including distal, proximal, collapsible, or self-expanding baskets); a conical configuration including forward-facing, reverse, or multi-layer conical meshes; an umbrella or parachute configuration (e.g., including retractable or dual-layer membrane umbrellas); a cylindrical or tubular configuration, including fixed, expandable, or porous polymer sleeves; a funnel or bell-shaped configuration, including dual-funnel or asymmetric funnel geometries; a mesh membrane or pouch configuration, including microporous or multi-zone filtration membranes; a cage or frame configuration, such as zigzag, diamond-shaped, or collapsible nitinol cages; a spiral or helical configuration; a multi-stage or basket-with-outlet configuration; a dual filter configuration providing proximal and distal protection; a retractable or collapsible configuration; a balloon-integrated configuration; a self-expanding stent-integrated configuration; an axially translatable or rotatable configuration; or a retrievable filter cartridge configuration. In some embodiments, two or more filters are combined with the stent device 100. For example, two or more filters can be positioned within the lumen of the stent device 100 sequentially and be, for example, configured to trap different type or sized emboli and other materials. The two or more filters can be of the same type or of different types.

In embodiments where the filter 800 is physically attached to the stent device 100, the cage or frame formed by the legs 802 of the filter can be an extension of the stent material which is formed to the desired shape. The filter 800 can also be attached or coupled with the stent device 100 by a welding or bonding process, or via the use of friction fitting with connectors or by designing the pieces that are to be connected such that they “snap” together and create a durable friction fit. In embodiments where the filter 800 is not physically attached to the stent device 100, the filter is implanted separately from the stent device 100. The filter 800 can be designed to have position stability via hooks or anchors, using the same concepts previously described for the stent itself. The position stability can provide migration resistance whether the filter is placed inside or within the stent or proximal or distal to it.

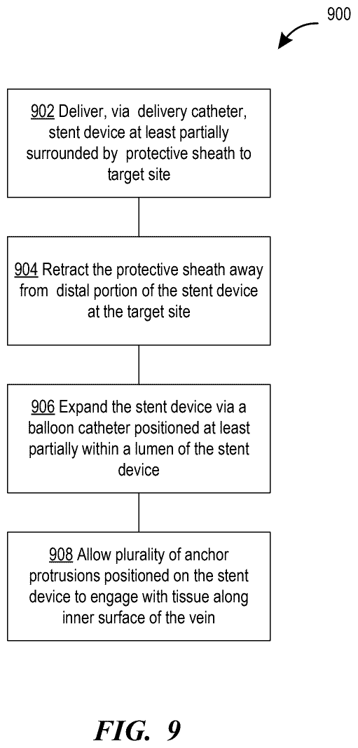

FIG. 9 is a flow chart of a method 900 for deploying a bioresorbable stent device (e.g., the stent device 100 in FIGS. 1A-1D) to a vessel (e.g., a vein) in accordance with some embodiments of the present technology. At 902, the method includes delivering, via a delivery catheter (e.g., a delivery catheter 306 in FIG. 3A), the stent device at least partially surrounded by a protective sheath (e.g., the sheath 308 in FIG. 3A) to a target site in a vein. At 904, the method includes at least partially retracting the protective sheath away from a distal portion of the stent device (e.g., a portion at an end of the stent device that is farthest away in a direction of delivering a catheter) at the target site. As shown in FIG. 3B, the sheath 308 has been removed, for example, by pulling the sheath 308 away. At 906, the method includes expanding the stent device via a balloon catheter (e.g., the balloon catheter 304) positioned at least partially within a lumen of the stent device. For example, expanding the stent device via the balloon catheter can include expanding the stent device from a delivery state having a first diameter below 4 millimeters to an expanded state having a second diameter of at least 10 millimeters. At 908, the method includes allowing a plurality of anchors protrusions (e.g., the anchor protrusions 104 in FIG. 1A) positioned on the stent device to engage tissue along an inner surface of the vein. The stent device can be repositioned from the target site to an additional site in the vein by at least partially collapsing the expanded stent device and moving the stent device, using the delivery catheter, to the additional site in the vein. The delivery catheter and the balloon catheter can be removed from inside by at least partially deflating the balloon catheter and pulling the delivery catheter and the balloon catheter away from the vein.

In some embodiments, the method includes in vivo customization of the stent device by cutting or otherwise removing portions of the stent device to change the overall length of the stent device and/or provide an opening (e.g., a fenestration) for transverse blood flow. For example, the stent devices disclosed herein can undergo in vivo scoring or electrolytic detachment procedures. In some embodiments, for example, a distal end of a delivery sheath and/or other delivery component can include a scoring (e.g., a cutting) feature. The scoring feature can be used for cutting the stent device by wrapping the scoring feature around a distal end of the stent device and causing the lumen of the stent device to collapse by the scoring feature. The scoring allows a user to shorten the length of the stent device to a desired length, and position the opening end of the stent device at a precise, desired location in the vein. As another example, the stent device includes an electrolytic detachment feature which comprises of a metal ring or a loop at the distal end of the delivery catheter or sheath. The electrolytic detachment feature is connected to a metal wire that traverses the length of the delivery catheter and is connected to a DC power source, for example by a wire. The electrolytic detachment feature and the wire are made of a conductive and non-corrosive material, such as gold or stainless steel. The other terminal of the DC power source is placed as a patch on the patient's skin. When the circuit is closed, a small electrical current is focused at the area of contact between the electrolytic detachment feature and the stent device, which triggers the process of electrolysis and thus rapid cutting of the stent at that location. In some embodiments, the stent devices disclosed herein can be cut using other suitable mechanisms known in the art.

A method for manufacturing a bioresorbable stent (e.g., the stent device 100 in FIGS. 1A-1D) can include forming a bioresorbable strut with controlled material properties. Alternatively, a wire can be used instead of a strut. The strut formation can include precision casting methods known in the art combined with extrusion and drawing processes under controlled thermal conditions. In order to form struts, the extrusion can include melt extrusion or solution spinning with a controlled cooling and solidification process. The surface of the bioresorbable strut can be treated to increase coating adhesion as well as to optimize the surface characteristics of the strut. For example, the surface of the bioresorbable strut can be etched (e.g., by acid etching) to form an additional oxide layer to promote adhesion of the strut (e.g., adhesion needed for coatings). Oxide formation at the strut surfaces has been experimentally confirmed by SEM imaging.

The method can include winding the bioresorbable strut into a tubular stent structure (e.g., the tubular frame 102 in FIG. 1A). The winding process can include precision control for achievement of appropriate positioning and tension. Degradation of the Mg alloy material can be prevented during the winding process with accurate temperature control. The final stent dimensions are controlled by application of mandrels and forms. To ensure consistency of the strut patterns, the winding process is monitored. The structure is stabilized with post-forming treatments. In some embodiments, the tubular stent structure can be formed by laser tube cutting, photochemical etching and forming, or other methods known in the art suitable for manufacturing Mg-alloy stents.

The method can further include applying at least one coating layer that covers at least a portion of the inner and/or outer surfaces of the stent (e.g., the coatings 122 in FIG. 1D). In some embodiments, the one or more coatings are applied to cover all surfaces of the stent. As described with respect to FIG. 1D, the one or more coatings can be engineered to cover specific portions of the stent. The strut surfaces can be treated with a primer for improved coating adhesion.

Applying the at least one coating can be performed using methods known in the art, such as a spin coating, dip coating, vapor deposition, and/or spray coating (e.g., ultrasonic spray) method. The at least one coating layer can include a coating using one or more graft materials. A graft material can promote rapid endothelialization while maintaining sufficient material presence to reinforce the stent structure. Materials contemplated for this graft component include polymers or fluoropolymers such as polyester or a fluoropolymer (e.g., fluorinated ethylene propylene (FEP), and polytetrafluoroethylene (PTFE)). The coatings can be post-processed by drying and/or curing.

The method can include forming anchor barbs (e.g., the anchor protrusions 104 in FIG. 1A) and coupling the anchor barbs on an outer surface of the tubular stent structure (e.g., with bioresorbable adhesives).

The method can further include forming at least one longitudinal spine (e.g., spine 426 in FIG. 4C) from the bioresorbable strut and attaching the longitudinal spine to the tubular stent structure. A spine can be coupled with an inner surface or an outer surface of the stent by bioresorbable adhesive materials, crimping mechanisms (e.g., looping a strut around the spine and a strut of the stent), marker bands, weaving patterns (e.g., the spine passing through the stent structure in an alternative manner), thermal bonding, and/or mechanical interlocking designs. The one or more coatings can be applied before or after the coupling of the anchor barbs and/or a spine.

In some embodiments, the stent can further be sterilized using, for example, ethylene oxide sterilization, radiation sterilization, and/or other sterilization approaches suitable for sterilizing sensitive bioresorbable materials.

In some embodiments, the various embodiments of the stent and stent systems of the present disclosure can be integrated with AI systems and methods for treatment planning, procedural visualization, post-implantation monitoring, and predictive analytics. Specifically, the AI-based methods and systems can be trained to predict treatment success, assist in planning stent implantation procedures, enhance visualization during the implantation process, provide ongoing monitoring of the stent status post-implantation, and generate actionable recommendations based on patient-specific data and predictive analytics.

An AI system can include a computer device (or system) with at least one memory and at least one processor for executing instructions stored at the memory. The system can further include or be in communication with a variety of data sources that can store patient data. The patient data can include imaging data (e.g., fluoroscopy, computed tomography (CT), magnetic resonance imaging (MRI), optical coherence tomography (OCT), and/or intravascular ultrasound (IVUS)), patient-specific physiological data, historical outcome data, blood flow measurement data, and/or biomarker data indicative of vascular healing and/or stent degradation. For example, the AI system can be incorporated within, or be in communication with, hospital information systems and picture archiving and communication systems (PACSs). The computer device can include an AI algorithm trained using historical patient data and outcomes to perform specific tasks. The AI algorithm can receive input data including from one or more of the various data sources and output desired predictions or analyses based on the input. In some embodiments, at least a part of the computation by the AI algorithm includes cloud computing and/or edge computing.

As an example, an AI system can determine stent degradation status and generate treatment recommendations based on multimodal patient data from imaging, hemodynamics, and biomarkers. OCT and IVUS patient data can be processed to quantify strut discontinuity, neointimal coverage, malposition, lumen geometry, and plaque burden, while blood flow measurement data (e.g., fractional flow reserve/instantaneous wave-free ration (FFR/iFR)) and computational fluid dynamics (CFD) metrics (e.g., wall shear stress and oscillatory shear index) provide information of functionality of the stent. Biomarker data (e.g., hs-CRP, IL-6, MMP-2/9, and troponin) can be processed to provide quantitative signals of inflammation, tissue remodeling, and myocardial injury. The AI system can evaluate, using the patient data, vascular healing status and detect stent degradation status. The AI system can further, for example, identify high-concern cases—e.g., elevated degradation, low healing, abnormal FFR, and raised inflammation indicators and generate recommended actions such as immediate high-resolution OCT to confirm focal fracture versus diffuse resorption, optimization of antiplatelet therapy, and targeted intervention (balloon optimization or thin-strut DES placement) with post-procedure FFR reassessment, followed by biomarker rechecks and repeat imaging to monitor recovery.

From the foregoing, it will be appreciated that specific embodiments of the invention have been described herein for purposes of illustration, but that various modifications may be made without deviating from the scope of the invention. Accordingly, the invention is not limited except as by the appended claims.

### EXAMPLE EMBODIMENTS

In accordance with some embodiments, a bioresorbable venous stent device includes a tubular frame having a first open end, a second open end, and a lumen extending between the first and second open ends. The frame includes a plurality of struts having a thickness between 75 micrometers and 450 micrometers or 100 micrometers and 300 micrometers. After implant in a vein, the tubular frame is configured to elastically deform. The plurality of struts include a Mg alloy having a concentration of Mg of at least 95 percentage weight and a maximum grain size of 10 micrometers. The tubular frame is configured to withstand at least 40 percent compression associated with dynamic movement of the vein. The device further includes an anchor protrusion coupled to at least one of the plurality of struts and extending outwardly away from the lumen. The anchor protrusion is configured to penetrate an inner wall of the vein to resist migration of the venous stent during and after implant. The bioresorbable venous stent has a degradation time of up to six months after being implanted within the vein.

In some embodiments, the bioresorbable venous stent device can include a plurality of struts that have a thickness between 125 micrometers and 150 micrometers.

In some embodiments, the bioresorbable venous stent device can include a configuration to expand from a delivery state to an expanded state. An expansion ratio between the expanded state and the delivery state can be greater than two, three, four, five, or six.

In some embodiments, the bioresorbable venous stent device can include a Mg alloy that has a concentration of Mg of at least 98 percentage weight.

In some embodiments, the bioresorbable venous stent device can include Mg that can have a maximum grain size of 1 micrometer.

In some embodiments, the bioresorbable venous stent device can include a Mg alloy that can include up to 5 percentage weight of manganese, zinc, and/or calcium.

In some embodiments, the bioresorbable venous stent device can include a tubular frame that has a first end portion proximate the first open end, a second end portion proximate the second open end, and a medial portion between the first and second end portions. The stent device can further include a plurality of radiopaque markers including a first radiopaque marker at the first end portion and a second radiopaque marker at the second end portion. The first and second markers can be radially offset from each other.

In some embodiments, the bioresorbable venous stent device can include a tubular frame that has eyelets. The radiopaque markers can have a conical shape that can define a base portion and a tip portion. The radiopaque markers can be positioned within the eyelets of the tubular frame so that the base portion of a respective radiopaque marker can be at least partially embedded within a respective eyelet and the tip portion of the respective radiopaque marker can extend away from the tubular frame. The tip portion of the respective radiopaque marker can be configured to project into the inner wall of the vein.

In some embodiments, the bioresorbable venous stent device can include an anchor protrusion that can include a curved or straight barb.

In some embodiments, at least a portion of the plurality of struts that can include a coating having multiple layers of a biodegradable polyester and/or a therapeutic agent.

In some embodiments, at least a portion of the plurality of struts can include a coating. The coating can have a first degradation speed, the Mg alloy can have a second degradation speed, and the first degradation speed is slower than the second degradation speed.

In some embodiment, at least a portion of the plurality of struts comprises a coating made of a non-resorbable material.

In some embodiments, the Mg alloy does not include rare earth metals.

In some embodiments, at least a portion of the plurality of struts that can include a coating having one or more layers of poly-L-lactic acid (PLLA), polylactic acid (PLA), polyglycolic acid (PGA), polycaprolactone (PCL), and/or a copolymer thereof.

In some embodiments, at least a portion of the plurality of struts that can include a coating having one or more layers of a therapeutic agent selected from antiproliferative drugs, anti-inflammatory agents, antithrombotic compounds, endothelialization-promoting factors, and/or growth factors.

In some embodiments, the bioresorbable venous stent device can include a degradation time that can range from one to three months, one to six months, three to six months, or three to four months from when the venous stent device is implanted inside the vein. In alternative embodiments, the degradation time can longer (e.g., three to twelve months, six to twelve months, nine to eighteen months, one to two years, or 1.5 to 3 years.

In some embodiments, the bioresorbable venous stent device can include a Mg alloy that can be treated during manufacture with cold drawing and heat treatment to increase the elasticity of the tubular frame.

In some embodiments, the bioresorbable venous stent device can include a tubular frame that can move from a delivery state having a first diameter below 4 millimeters to an expanded state having a second diameter of at least 10 millimeters.

In some embodiments, the bioresorbable venous stent device can include a longitudinal spine positioned between adjacent struts and extending in a longitudinal direction at least partially between a first end region and a second end region.

In some embodiments, the bioresorbable venous stent device can include a filter coupled to the tubular frame and positioned at least partially within the lumen. The filter can be configured to trap emboli.

In some embodiments, the bioresorbable venous stent device can include struts that can form patterns having peak regions. The struts can be connected to each other at the peak regions. The peak regions can define an angle of at least 30 degrees between adjacent struts.

In some embodiments, the bioresorbable venous stent device can include struts that can form patterns having peak regions. The struts can be connected to each other at the peak regions. The peak regions can have a rounded shape that can be configured to reduce stress at the peak regions.

In accordance with some embodiments, a bioresorbable stent device includes a tubular frame having a first open end, a second open end, and a lumen extending between the first and second open ends. The frame includes a plurality of struts having a thickness between 100 micrometers and 300 micrometers. After implant in a vessel, the tubular frame is configured to elastically deform. The tubular frame is configured to withstand at least 40 percent compression associated with dynamic movement of the vessel. The plurality of struts include a Mg alloy having a concentration of Mg of at least 95 percentage weight and a maximum grain size of 10 micrometers. At least a portion of the plurality of struts includes a coating having multiple layers of a biodegradable polyester and/or a therapeutic agent. The device further includes an anchor protrusion coupled to at least one of the plurality of struts and extending outwardly away from the lumen. The anchor protrusion is configured to engage (e.g., at least partially penetrate) an inner wall of the vessel to resist migration of the stent device during and after implant. The bioresorbable stent device has a degradation time ranging from three to six months after being implanted within the vessel.

In accordance with some embodiments, a method of delivering a bioresorbable venous stent device to a vein includes delivering, via a delivery catheter, the stent device at least partially surrounded by a protective sheath to a target site in a vein. A tubular frame has a first open end, a second open end, and a lumen extending between the first and second open ends, and the frame includes a plurality of struts having a thickness between 75 micrometers and 450 micrometers. The plurality of struts include a Mg alloy having a concentration of Mg of at least 95 percentage weight and a maximum grain size of 10 micrometers. The tubular frame is configured to withstand at least 40 percent compression associated with dynamic movement of the vein. The method further includes at least partially retracting the protective sheath away from a distal portion of the stent device at the target site, expanding the stent device via a balloon catheter positioned at least partially within a lumen of the stent device, and allowing a plurality of anchor protrusions positioned on the stent device to engage tissue along an inner surface of the vein. The stent device has a degradation time ranging from three to six months when implanted inside the vein.

In some embodiments, the method can include a protective sheath with a distal end that includes a scoring feature. The method can include cutting the stent device by wrapping the scoring feature around a distal end of the stent device. The method can include causing the lumen of the stent device to collapse by the scoring feature.

In some embodiments, the method can include repositioning the stent device from the target site to an additional site in the vein by at least partially collapsing the expanded stent device and moving the stent device, using the delivery catheter, to the additional site in the vein.

In some embodiments, the method can include a balloon catheter that includes a first balloon section and a second balloon section that can be independently inflated. Expanding the stent device via the balloon catheter can include expanding a first portion of the stent device using the first balloon section to a first expanded diameter. Expanding the stent device via the balloon catheter can include expanding a second portion of the stent device using the second balloon section to a second expanded diameter that can be different from the first expanded diameter.

In some embodiments, the method can include expanding the stent device via the balloon catheter to expand the stent device from a delivery state having a first diameter below 4 millimeters to an expanded state having a second diameter of at least 10 millimeters.

In accordance with one embodiments, a bioresorbable stent device includes a tubular frame having a first open end, a second open end, and a lumen extending between the first and second open ends. The frame includes a plurality of struts having a thickness between 75 micrometers and 450 micrometers. After deployment in a vessel, the tubular frame is configured to elastically deform. The plurality of struts can include a magnesium alloy having a concentration of magnesium of at least 90 percentage weight (e.g., 95%) and a maximum grain size 10 micrometers. The tubular frame can be configured to withstand at least 40 percent compression associated with dynamic movement of the vessel. The bioresorbable stent can have a degradation time up to six months or shorter (e.g. up to 2 months). In some embodiments the degradation time may be longer than 6 months (e.g., up to one year or over one year).

In some embodiments, the stent device further includes a plurality of anchor elements coupled to the tubular frame and configured to contact an inner wall of the vessel to resist migration of the tubular frame after implant. In some embodiments, the anchor protrusion or elements are configured to press against the inner wall of the vessel without penetrating the inner wall. In some embodiments the anchor elements penetrate the inner wall and/or other tissue.

In some embodiments, the tubular frame can include one or more struts having a non-uniform width and/or a non-uniform thickness along a strut length. In some embodiments, the tubular frame can have a predefined non-straight shape of stent structures (in a deployed state) and varying link attachment points. In some embodiments, stent devices can include various markers embedded in, carried by, and/or otherwise incorporated into the stent frame, and/or may include an insulative coating on the marker itself.

### CONCLUSION

The above detailed description of embodiments of the technology are not intended to be exhaustive or to limit the technology to the precise form disclosed above. Although specific embodiments of, and examples for, the technology are described above for illustrative purposes, various equivalent modifications are possible within the scope of the technology as those skilled in the relevant art will recognize. For example, although steps are presented in a given order, alternative embodiments can perform steps in a different order. The various embodiments described herein can also be combined to provide further embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms can also include the plural or singular term, respectively.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications can be made without deviating from the technology. Further, while advantages associated with some embodiments of the technology have been described in the context of those embodiments, other embodiments can also exhibit such advantages, and not all embodiments need necessarily exhibit such advantages to fall within the scope of the technology. Accordingly, the disclosure and associated technology can encompass other embodiments not expressly shown or described herein.

We claim:

1. A bioresorbable venous stent device comprising:
a tubular frame having a first open end, a second open end, and a lumen extending between the first and second open ends, the frame comprising a plurality of struts having a thickness between 75 micrometers and 450 micrometers, wherein—
after implant in a vein, the tubular frame is configured to elastically deform,
the plurality of struts comprise a magnesium alloy having a concentration of magnesium of at least 95 percentage weight and a maximum grain size 10 micrometers, and
the tubular frame is configured to withstand at least 40 percent compression associated with dynamic movement of the vein; and
an anchor protrusion coupled to at least one of the plurality of struts and extending outwardly away from the lumen, wherein the anchor protrusion is configured to engage an inner wall of the vein to resist migration of the venous stent device during and after implant,
wherein the bioresorbable venous stent device has a degradation time up to six months after implanted within the vein.

2. The bioresorbable venous stent device of claim 1 wherein the plurality of struts have a thickness between 125 micrometers and 150 micrometers.

3. The bioresorbable venous stent device of claim 1 wherein—
the venous stent device is configured to expand from a delivery state to an expanded state, and
an expansion ratio between the expanded state and the delivery state is greater than two.

4. The bioresorbable venous stent device of claim 1 wherein the magnesium alloy has a concentration of magnesium of at least 95 percentage weight.

5. The bioresorbable venous stent device of claim 1 wherein the magnesium has a maximum grain size 1 micrometers.

6. The bioresorbable venous stent device of claim 1 wherein the magnesium alloy further comprises up to 5 percentage weight of manganese, zinc, and/or calcium.

7. The bioresorbable venous stent device of claim 1 wherein:
the tubular frame has a first end portion proximate the first open end, a second end portion proximate the second open end, and a medial portion between the first and second end portions; and
the stent device further comprises a plurality of radiopaque markers including a first radiopaque marker at the first end portion and a second radiopaque marker at the second end portion, wherein the first and second markers are radially offset from each other.

8. The bioresorbable venous stent device of claim 7 wherein—
the tubular frame has eyelets,
the radiopaque markers have a conical shape defining a base portion and a tip portion,
the radiopaque markers are positioned within the eyelets of the tubular frame so that the base portion of a respective radiopaque marker is at least partially embedded within a respective eyelet and the tip portion of the respective radiopaque marker extends away from the tubular frame, and
the tip portion of the respective radiopaque marker is configured to project into the inner wall of the vein.

9. The bioresorbable venous stent device of claim 1 wherein the anchor protrusion comprises a curved or straight barb.

10. The bioresorbable venous stent device of claim 1 wherein at least a portion of the plurality of struts comprises a coating having multiple layers of a biodegradable polyester and/or a therapeutic agent.

11. The bioresorbable venous stent device of claim 1 wherein at least a portion of the plurality of struts comprise a coating having one or more layers of poly-L-lactic acid (PLLA), polylactic acid (PLA), polyglycolic acid (PGA), polycaprolactone (PCL), and/or a copolymer thereof.

12. The bioresorbable venous stent device of claim 1 wherein at least a portion of the plurality of struts comprise a coating having one or more layers of a therapeutic agent selected from antiproliferative drugs, anti-inflammatory agents, antithrombotic compounds, endothelialization-promoting factors, and/or growth factors.

13. The bioresorbable venous stent device of claim 1 wherein—
at least a portion of the plurality of struts comprises a coating,
the coating has a first degradation speed,
the Mg alloy has a second degradation speed, and
the first degradation speed is slower than the second degradation speed.

14. The bioresorbable venous stent device of claim 1 wherein at least a portion of the plurality of struts comprises a coating made of a non-resorbable material.

15. The bioresorbable venous stent device of claim 1 wherein the magnesium alloy does not include rare earth metals.

16. The bioresorbable venous stent device of claim 1 wherein the degradation time ranges from three to four months when the venous stent device is implanted inside the vein.

17. The bioresorbable venous stent device of claim 1 wherein the magnesium alloy is treated during manufacture with cold drawing and heat treatment to increase the elasticity of the tubular frame.

18. The bioresorbable venous stent device of claim 1 wherein the tubular frame is configured to move from a delivery state having a first diameter below 4 millimeters to an expanded state having a second diameter of at least 10 millimeters.

19. The bioresorbable venous stent device of claim 1, further comprising a longitudinal spine positioned between adjacent struts and extending in a longitudinal direction at least partially between a first end region and a second end region.

20. The bioresorbable venous stent device of claim 1, further comprising a filter coupled to the tubular frame and positioned at least partially within the lumen, wherein the filter is configured to trap emboli.

21. The bioresorbable venous stent device of claim 1 wherein—
the struts form patterns having peak regions,
the struts are connected to each other at the peak regions, and
the peak regions define an angle of at least 30 degrees between adjacent struts.

22. The bioresorbable venous stent device of claim 1 wherein at least one of the struts has a non-uniform width and/or a non-uniform thickness along a strut length.

23. The bioresorbable venous stent device of claim 1 wherein—
the struts form patterns having peak regions,
the struts are connected to each other at the peak regions, and
the peak regions have a rounded shape configured to reduce stress at the peak regions.

24. The bioresorbable venous stent device of claim 1 wherein the anchor protrusion is configured to penetrate the inner wall of the vein.

25. The bioresorbable venous stent device of claim 1 wherein the anchor protrusion is configured to press against the inner wall of the vein without penetrating the inner wall.

26. A bioresorbable stent device comprising:
a tubular frame having a first open end, a second open end, and a lumen extending between the first and second open ends, the frame comprising a plurality of struts having a thickness between 75 micrometers and 450 micrometers, wherein—
after implant in a vessel, the tubular frame is configured to elastically deform,
the tubular frame is configured to withstand at least 40 percent compression associated with dynamic movement of the vessel,
the plurality of struts comprise a magnesium alloy having a concentration of magnesium of at least 95 percentage weight and a maximum grain size 10 micrometers, and
at least a portion of the plurality of struts comprises a coating having multiple layers of a biodegradable polyester and/or a therapeutic agent; and
an anchor protrusion coupled to at least one of the plurality of struts and extending outwardly away from the lumen, wherein the anchor protrusion is configured to engage an inner wall of the vessel to resist migration of the stent device during and after implant,
wherein the bioresorbable stent device has a degradation time up to six months after being implanted within the vessel.

27. A method of delivering a bioresorbable venous stent device to a vein, the method comprising:
delivering, via a delivery catheter, the stent device at least partially surrounded by a protective sheath to a target site in a vein, wherein—
a tubular frame has a first open end, a second open end, and a lumen extending between the first and second open ends, the frame comprising a plurality of struts having a thickness between 75 micrometers and 450 micrometers, and
the plurality of struts comprise a magnesium alloy having a concentration of magnesium of at least 95 percentage weight and a maximum grain size of 10 micrometers, and
the tubular frame is configured to withstand at least 40 percent compression associated with dynamic movement of the vein;
at least partially retracting the protective sheath away from a distal portion of the stent device at the target site;
expanding the stent device via a balloon catheter positioned at least partially within a lumen of the stent device; and
allowing a plurality of anchor protrusions positioned on the stent device to engage with tissue along an inner surface of the vein,
wherein the stent device has a degradation time ranging from three to six months when the stent device is implanted inside the vein.

28. The method of claim 27 wherein—
a distal end of the protective sheath comprises a scoring feature, and
the method comprises cutting the stent device by wrapping the scoring feature around a distal end of the stent device and causing the lumen of the stent device to collapse by the scoring feature.

29. The method of claim 27, further comprising repositioning the stent device from the target site to an additional site in the vein by at least partially collapsing the expanded stent device and moving the stent device, using the delivery catheter, to the additional site in the vein.

30. The method of claim 27, wherein—
the balloon catheter comprises a first balloon section and a second balloon section that are independently inflated, and
expanding the stent device via the balloon catheter comprises expanding a first portion of the stent device using the firsts balloon section to a first expanded diameter and expanding a second portion of the stent device using the second balloon section to a second expanded diameter that is different from the first expanded diameter.

31. The method of claim 27, wherein expanding the stent device via the balloon catheter expands the stent device from a delivery state having a first diameter below 4 millimeters to an expanded state having a second diameter of at least 10 millimeters.

32. A bioresorbable stent device comprising:
a tubular frame having a first open end, a second open end, and a lumen extending between the first and second open ends, the frame comprising a plurality of struts having a thickness between 75 micrometers and 450 micrometers, wherein—
after deployment in a vessel, the tubular frame is configured to elastically deform,
the plurality of struts comprise a magnesium alloy having a concentration of magnesium of at least 90 percentage weight and a maximum grain size 10 micrometers,
the tubular frame is configured to withstand at least 40 percent compression associated with dynamic vessel movement, and
the bioresorbable stent has a degradation time up to six months.

33. The bioabsorbable stent device of claim 32 further comprising a plurality of anchor elements coupled to the tubular frame and configured to contact an inner wall of the vessel to resist migration of the tubular frame after implant.
